(19) United States
(12) Patent Application Publication
(10) Pub. No.: US 2008/0039496 A1
(43) Pub. Date: Feb. 14, 2008

(54) Method of Modulating Neurite Outgrowth by the use of a Galanin-3 Receptor Antagonist

(76) Inventors: Thomas P. Blackburn, Hoboken, NJ (US); Roderick E.M. Scott, Pelham, NY (US)

Correspondence Address: HELLER EHRMAN LLP, 275 MIDDLEFIELD ROAD, MENLO PARK, CA, 94025-3506, US

(21) Appl. No.: 11/768,889

(22) Filed: Jun. 26, 2007

Related U.S. Application Data
(60) Provisional application No. 60/816,682, filed on Jun. 26, 2006.

(51) Int. Cl. A61K 31/404; A61K 31/415; A61K 31/428; A61K 31/4439; A61K 31/47; A61P 25/00

(52) U.S. Cl. 514/314; 514/339; 514/367; 514/406; 514/414; 514/418

(57) ABSTRACT

The present invention provides methods of modulating neurite outgrowth in an animal. The methods comprise a general administration of galanin-3 receptor antagonists under conditions sufficient to produce neurite outgrowth.

### CROSS-REFERENCE TO RELATED APPLICATIONS

This is a non-provisional application filed under 37 CFR 1.53(b), claiming priority under USC Section 119(e) to provisional Application Ser. No. 60/816,682, filed Jun. 26, 2006 which is incorporated by reference herein in its entirety.

### BACKGROUND OF THE INVENTION

Galanin is a 29 to 30 amino acid containing neuropeptide involved in a variety of peripheral and central physiological and pathological processes, including gastrointestinal motility, cardiovascular contraction, neuroendocrine function, feeding behavior, pain perception, learning, memory, anxiety and depression. The neuropeptide Galanin mediates its effects through three known G-protein coupled receptor subtypes GalR1, GalR2 and GalR3, and has been implicated in many physiological processes including feeding behavior, pain and depression. Central Galanin-3 receptor (GalR3) mRNA distribution is discrete with a prominent representation in the hypothalamus and lower levels in some limbic regions including the locus ceuleus, the dorsal raphe and the midbrain central gray.

Several studies have demonstrated the ability of Galanin to modulate the central 5-hydroxytryptamine (5-HT) function (Fuxe et al. Ann N Y Acad Sci. 1998 Dec. 21; 863:274-90; Kehr et al. Neuropsychopharmacology. 2002 September; 27(3):341-56; Yoshitake et al. Neurosci Lett. 2003 Mar. 27; 339(3):239-42). HT-2157, a selective GalR3 antagonist, has been shown to antagonize the inhibitory effect of Galanin on 5-HT transmission (Rowley et al. Br J Pharmacol 2005, Winter Meeting, P125) and therefore to increase extracellular levels of 5-HT in various brain regions (Rowley et al. Br J Pharmacol 2005, Winter Meeting, P127).

Citation of any document is not intended as an admission that it is pertinent prior art. All statements as to the date or representation as to the contents of these documents is based on the information available to the applicant and does not constitute any admission as to the correctness of the dates or contents of the documents.

### SUMMARY OF THE INVENTION

The present invention relates to administration of galanin-3 receptor (GalR3) antagonists to modulate neurite outgrowth.

In one embodiment the method is directed to the modulation of neurite outgrowth by the administration of a galanin-3 receptor antagonist to an animal. In one embodiment the neurite outgrowth is enhanced or increased by the administration of a galanin-3 receptor antagonist to an animal relative to normal growth in the absence of the galanin-3 receptor antagonist.

In another embodiment the method is directed to treating a subject in need of treatment for a nerve cellular injury and/or trauma which comprises administering to the subject galanin-3 receptor antagonist.

In one embodiment the method is directed to treating a subject in need of treatment for a nerve cellular injury and/or trauma which comprises administering to the subject an amount of galanin-3 receptor antagonist effective to treat the subject's nerve injury or trauma, wherein the galanin-3 receptor antagonist has the structure: 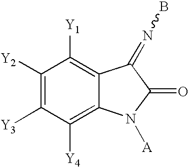
wherein each of Y₁, Y₂, Y₃, and Y₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, or C₅-C₇ cycloalkenyl; —F, —Cl, —Br, or —I; —NO₂; —N₃; —CN; —OR₄, —SR₄, —OCOR₄, —COR₄, —NCOR₄, —N(R₄)₂, —CON(R₄)₂, or —COOR₄; aryl or heteroaryl; or any two of Y₁, Y₂, Y₃ and Y₄ present on adjacent carbon atoms can constitute a methylenedioxy group;
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl;
wherein A is A′, straight chained or branched C₁-C₇ alkyl, aryl, heteroaryl, aryl(C₁-C₆)alkyl or heteroaryl(C₁-C₆)alkyl;
wherein A′ is 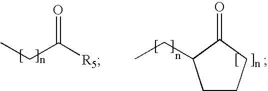
wherein R₁ and R₂ are each independently —H, straight chained or branched C₁-C₇ alkyl, —F, —Cl, — 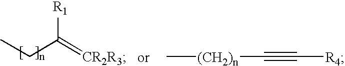
Br, —I, —NO₂, or —CN;
wherein R₃ is —H, straight chained or branched C₁-C₇ alkyl, —F, —Cl, —Br, —I, —NO₂, —CN, —OR₆, aryl or heteroaryl;
wherein R₅ is straight chained or branched C₁-C₇ alkyl, —N(R₄)₂, —OR₆ or aryl;
wherein R₆ is straight chained or branched C₁-C₇ alkyl or aryl;
wherein B is aryl, or heteroaryl; provided however, if B is aryl or heteroaryl the carbon atom or carbon atoms ortho to the nitrogen atom of the imine bond may only be substituted with one or more of the following: —H, —F, —Cl, —Br, —I, —CN, methyl, ethyl or methoxy;
wherein each n is independently an integer from 1 to 4 inclusive;
wherein the compound is a pure Z imine isomer, a pure E imine isomer, or a mixture of Z and E imine isomers;
or a pharmaceutically acceptable salt thereof.

The present invention also provides a method of treating a subject in need of treatment for a nerve cellular injury and/or nerve trauma which compromises administering to the subject an effective amount of galanin-3 receptor antagonist, wherein the galanin-3 receptor antagonist has the structure: 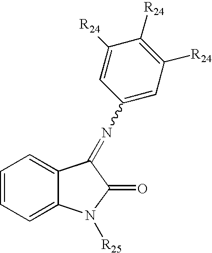
wherein each R₂₄ is independently one or more of the following: H, F, Cl, Br, I, CF₃ or OCH₃;
wherein R₂₅ is methyl, ethyl, allyl or phenyl and the phenyl is optionally substituted with a F, Cl, Br, CF₃, or OR₄; and
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl.

The present invention also provides a method of treating a subject in need of treatment for a nerve cellular injury and/or trauma which compromises administering to the subject an effective amount of a galanin-3 receptor antagonist compound, wherein the a galanin-3 receptor antagonist compound has the structure: 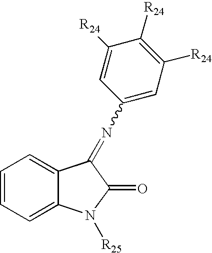
wherein each R₂₄ is independently one or more of the following: H, F, Cl, Br, I, CF₃ or OCH₃;
wherein R₂₅ is methyl, ethyl, allyl or phenyl and the phenyl is optionally substituted with a F, Cl, Br, CF₃, or OR₄; and
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl.

As described herein, the administration of galanin-3 receptor (Gal3R) antagonist compounds can be done alone or with the administration of other compounds for example benzodiazepine or selective serotonin reuptake inhibitors (SSRI).

It is contemplated that in the various embodiments, the Gal-3 receptor antagonist is HT-2157 (1,3-dihydro-1-phenyl-3[[3-trifluoromethyl)phenyl]imino]-2H-indol-2-one; CAS No. 303149-14-6. 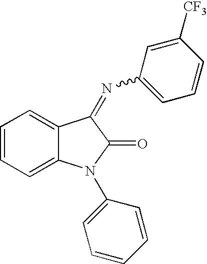
the E/Z isomers or mixtures thererof.

The present invention provides a method for treating inhibiting or ameliorating the effects of injuries or diseases that result in neuronal degeneration or a method for promoting neurogenesis. These methods involve administering to a patient in need thereof an effective amount of at least one indolone. It has been found the indolones of the present invention promote neurite outgrowth and neurogenesis.

Alternatively, the at least one indolone of the present invention is used to treat stem cells or neuronal progenitor cells prior to the cells being administered to the patient by implantation at the site of neuronal degeneration. The method of the present invention which promotes neurogenesis is involved in cell renewal in the central nervous system (CNS) and includes all types of CNS cells.

An embodiment of the present invention is used to treat primary nervous system injury e.g. closed head injuries and blunt trauma, including but not limited to those caused by participation in dangerous sports, penetrating trauma, including but not limited to those caused by gunshot wounds, hemorrhagic stroke, ischemic stroke, glaucoma, cerebral ischemia, or damages including but not limited to those caused by surgery such as tumor excision. The compounds of the invention may promote nerve regeneration in order to enhance or accelerate the healing of such injuries. In addition, the method may be used to treat, inhibit or ameliorate the effects of disease or disorder that results in a degenerative process.

An embodiment of the present invention a method of administration of a galanin-3 receptor antagonist to inhibit secondary degeneration which may otherwise follow primary nervous system injury.

The compounds of the invention may be used to treat various diseases or disorders of the central or peripheral nervous system, including but not limited to diabetic neuropathy, amyotrophic lateral sclerosis (ALS). The compounds of the invention may be used to treat peripheral nerve injuries and peripheral or localized neuropathies including, but not limited to, porphyria, acute sensory neuropathy, chronic ataxic neuropathy, complications of various drugs and toxins, amyloid polyneuropathies, adrenomyeloneuropathy, giant axonal neuropathy may be treated by this method.

In addition the compounds can be used for post-operative treatments such as for tumor removal from CNS and other forms of surgery on the CNS. The compounds can be used for treatment of spinal chord trauma.

In another embodiment, the invention is directed to a kit for the treatment of neural cellular injury and/or trauma comprising a galanin-3 receptor antagonist.

Other examples of Galanin 3 receptor antagonists can be found in U.S. Publication No. US2003/078271A1; and International Publication No. WO2004/093789 which are incorporated by reference.

### BRIEF DESCRIPTION OF THE DRAWINGS

FIG. 1 shows the plots of 4 output features generated from quantitative analysis of images of NS-1 cells by the Extended Neurite Outgrowth BioApplication. The data plotted is the mean value of each feature ±standard deviation from 2 wells per concentration of the compound. The definitions for the output features are as follows:

Neurite count: the number of neurites associated with the selected neurons.

Total neurite length: the total length of neurites for a selected neuron.

Average neurite length: the total neurite length divided by the neurite count for the selected neurons.

Branch point: the junction of three neurite segments.

FIG. 2 shows the qPCR analysis of the effects on Hes5 expression by HT-2157 treatment in NS-1 cells. The data plotted is the mean value of the relative RNA level of the cells in 2 wells ±standard deviation.

FIG. 3 shows the average neurite length analyzed by the Neurite Outgrowth BioApplication from the images of the mouse hippocampal neurons. The data plotted is the mean value ±standard deviation from 2 wells per concentration of the compound.

FIG. 4 is a photograph of a Western blot showing the effect of HT-2157 on GalR3 expression in Neuroscreen 1 (NS1) cells as performed by Western blot analysis of GalR3 expression in NS1 cells 24 hours after treatment with vehicle (V), HT-2157.

FIG. 5 is a chart showing the effect of HT-2157 treatment on the expression of Hes5 in NS1 cells by qPCR analysis of Hes5 expression in NS1 cells. NS1 cells were treated with Vehicle (Veh) or HT-2157 for 2 hours, 4 hours, or 24 hours.

FIG. 6A shows the effect of Hes5 knockdown by siRNA on neurite outgrowth in NS1 cells. Neurite length in untreated NS1 cells, and in NS1 cells treated with Vehicle, control siRNA, Hes5 siRNA. FIG. 6B shows neurite branch points in untreated NS1 cells, and in NS1 cells treated with Vehicle, control siRNA, Hes5 siRNA.

FIG. 7 shows the effect of HT-2157 on neurite outgrowth in NS1 cells. Data are representative of the mean +/− the stdev of two experiments. For each experiment, neurite outgrowth in a minimum of 100 cells was measured. Quantification of the effect of HT-2157 on neurite outgrowth in NS1 cells. 3 μM and 10 μM HT-2157 facilitates neurite outgrowth as indicated by an increase in: i) the number of neurites per cell (neurite count), ii) the total neurite length per cell, iii) the average neurite length per cell, iv) the number of neurite branch points per cell.

FIG. 8 shows the effect of HT-2157 on mRNA expression of the neurotrophins brain derived neurotrophic factor (BDNF) and nerve growth factor β (NGFb), and on expression of Hes5 in cultured mouse hippocampal neurons. The mean ±stdev of 2 experimental replications are shown. FIG. 8A shows the effect of 10 μM HT-2157 on BDNF expression. FIG. 8B shows the effect of 10 μM HT-2157 on NGFβ expression. FIG. 8C shows the effect of 10 μM HT-2157 on Hes5 expression.

FIG. 9 shows the effect of NGFβ on neurite outgrowth in cultured mouse hippocampal neurons. The mean ±sem of 8 experimental replications are shown. For each experiment, neurite outgrowth in a minimum of 100 cells was measured. Hippocampal neurons were treated with 100 ng/ml NGFβ for 24 hours and neurite growth measured in the Cellomics Arrayscan II. NGFβ enhances neurite outgrowth as evident by increased number of neurites per cell, increased neurite length, and increased branch points.

FIG. 10 shows the quantification of the effect of HT-2157 on neurite outgrowth in cultured hippocampal neurons. The mean ±sem of 8 experimental replications (96-wells) per drug dose and 16 replication per vehicle are shown. For each experiment, neurite outgrowth in a minimum of 100 cells was measured. FIG. 10A shows the quantification of the effects of HT-2157 on neurite outgrowth in hippocampal neurons as determined by the effect on neurite number per cell. FIG. 10B shows the quantification of the effects of HT-2157 on neurite outgrowth in hippocampal neurons as determined by the effect on the total neurite length per cell. FIG. 10C shows the quantification of the effects of HT-2157 on neurite outgrowth in hippocampal neurons as determined by the effect on neurite branch points per cell.

### DETAILED DESCRIPTION OF THE INVENTION

A growing body of evidence suggests that neurons continue to proliferate in the adult brain (Arsenijevic, Y. et al., Exp. Neurol., 170: 48-62 (2001); Vescovi, A. L. et al., Biomed. Pharmacother., 55:201-205 (2001); Cameron, H. A. and McKay, R. D., J. Comp. Neurol., 435:406-417 (2001); and Geuna, S. et al., Anat. Rec., 265:132-141 (2001)). Experimental strategies now are underway to transplant neuronal stem into adult brain for various therapeutic indications (Kurimoto, Y. et al., Neurosci. Lett., 306:57-60 (2001); Singh, G., Neuropathology, 21:110-114 (2001); and Cameron, H. A. and McKay, R. D., Nat. Neurosci., 2:894-897 (1999)). Much already is known about neurogenesis in embryonic stages of development (Saitoe, M. and Tully, T., “Making connections between synaptic and behavioral plasticity in Drosophila”, In Toward a Theory of Neuroplasticity, J. McEachem and C. Shaw, Eds. (New York: Psychology Press.), pp. 193-220 (2000)). Neuronal differentiation, neurite extension and initial synaptic target recognition all appear to occur in an activity-independent fashion.

Recent studies show that the activation of 5-HT1A receptor increases hippocampal neurogenesis (Santarelli et al. Science. 2003 Aug. 8; 301(5634):805-9.) and neurite outgrowth (Fricker et al. Brain Res Mol Brain Res. 2005 Aug. 18; 138(2):228-35). In this study, the effects of HT-2157 on enhancing neurite outgrowth were examined and the mechanisms underlying the modulation of neurite outgrowth were explored in both a PC12 sub-clone and primary mouse neuronal cultures. The results demonstrated that HT-2157 significantly enhanced neurite outgrowth of PC12 cells and primary mouse neurons. In addition HT-2157 down regulated the expression of Hes5, a vertebrate homologue of the Drosophila basic helix-loop-helix (bHLH) protein Hairy, which is known to be a transcriptional repressor that negatively regulates neuronal differentiation. Taken together, these findings indicate that the enhancement of neurite outgrowth by HT-2157 is mediated through the control of neuronal differentiation progression.

As used herein, the term “animal” or “subject” includes mammals, as well as other animals, vertebrate and invertebrate (e.g., birds, fish, reptiles, insects (e.g., Drosophila species), mollusks (e.g., Aplysia). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and primates (e.g., monkeys, chimpanzees), rodents (e.g., rats, mice, guinea pigs) and ruminents (e.g., cows, pigs, horses).

The animal or subject can be an animal with some form and degree of neurite impairment.

The term “stem cell” or neural stem cell (NSC)) as used herein, refers to an undifferentiated cell that is capable of self-renewal and differentiation into neurons, astrocytes and/or oligodendrocytes.

The term “progenitor cell” (e.g. neural progenitor cell) as used herein refers to a cell derived from a stem cell that is not itself a stem cell. Some progenitor cells can produce progeny that are capable of differentiating into more than one cell type.

As used herein “treating” includes prevention, amelioration, alleviation and/or elimination of the disease, disorder or condition being treated or one or more symptoms of the disease, disorder or condition being treated as well as improvement in the overall well being of a patient as measured by objective and/or subjective criteria. In some embodiments, treating is used for reversing, attenuating, minimizing, suppressing, or halting undesirable or deleterious effects of or effects from the progression of a disease, disorder or condition of the central and/or peripheral nervous system. In other embodiments the method of treating may be advantageously used in cases where additional neurogenesis or neurite outgrowth would replace, replenish or increase the number of cells lost due to injury or disease.

The present invention relates to administration of galanin-3 receptor (GalR3) antagonists to modulate neurite outgrowth.

In one embodiment the method is directed to the modulation of neurite outgrowth by the administration of a galanin-3 receptor antagonist to an animal. In one embodiment the neurite outgrowth is enhanced or increased by the administration of a galanin-3 receptor antagonist to an animal relative to normal growth in the absence of the galanin-3 receptor antagonist.

In another embodiment the method is directed to treating a subject in need of treatment for a nerve cellular injury and/or trauma which comprises administering to the subject galanin-3 receptor antagonist.

In one embodiment the method is directed to treating a subject in need of treatment for a nerve cellular injury and/or trauma which comprises administering to the subject an amount of galanin-3 receptor antagonist compound effective to treat the subject's nerve injury or trauma, wherein the galanin-3 receptor antagonist compound has the structure: 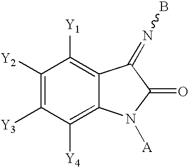
wherein each of Y₁, Y₂, Y₃, and Y₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, or C₅-C₇ cycloalkenyl; —F, —Cl, —Br, or —I; —NO₂; —N₃; —CN; —OR₄, —SR₄, —OCOR₄, —COR₄, —NCOR₄, —N(R₄)₂, —CON(R₄)₂, or —COOR₄; aryl or heteroaryl; or any two of Y₁, Y₂, Y₃ and Y₄ present on adjacent carbon atoms can constitute a methylenedioxy group;
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl;
wherein A is A′, straight chained or branched C₁-C₇ alkyl, aryl, heteroaryl, aryl(C₁-C₆)alkyl or heteroaryl(C₁-C₆)alkyl;
wherein A′ is 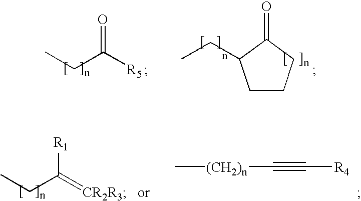
wherein R₁ and R₂ are each independently H, straight chained or branched C₁-C₇ alkyl, —F, —Cl, —Br, —I, —NO₂, or —CN;
wherein R₃ is H, straight chained or branched C₁-C₇ alkyl, —F, —Cl, —Br, —I, —NO₂, —CN, —OR₆, aryl or heteroaryl;
wherein R₅ is straight chained or branched C₁-C₇ alkyl, —N(R₄)₂, —OR₆ or aryl;
wherein R₆ is straight chained or branched C₁-C₇ alkyl or aryl;
wherein B is aryl, or heteroaryl; provided however, if B is aryl or heteroaryl the carbon atom or carbon atoms ortho to the nitrogen atom of the imine bond may only be substituted with one or more of the following: —H, —F, —Cl, —Br, —I, —CN, methyl, ethyl or methoxy;
wherein each n is independently an integer from 1 to 4 inclusive;
wherein the compound is a pure Z imine isomer, a pure E imine isomer, or a mixture of Z and E imine isomers;

In the present invention, the term “straight chained or branched C₁-C₇ alkyl” refers to a saturated hydrocarbon moiety having from one to seven carbon atoms inclusive. Examples of such substituents include, but are not limited to, methyl, ethyl, 1-propyl, 2-propyl, 1-butyl, 2-butyl, 2-methyl-2-propyl and 2-methyl-1-propyl. The term “C₂-C₇ alkenyl” refers to a mono-unsaturated hydrocarbon moiety having from two to seven carbon atoms inclusive. Examples of such substituents include, but are not limited to, ethenyl, prop-1-en-1-yl, prop-1-en-2-yl, prop-2-en-1-yl, but-3-en-2-yl and hept-2-en-1-yl. The term “C₃-C₇ alkynyl” refers to a hydrocarbon moiety having from three to seven carbon atoms and containing one carbon-carbon triple bond. Examples of such substituents include, but are not limited to, prop-1-ynyl, prop-2-ynyl, pent-2-ynyl, 4,4-dimethylpent-2-ynyl, 5-methylhex-3-yn-2-yl and hept-3-ynyl.

As used in the present invention, the term “cycloalkyl” includes C₃-C₇ cycloalkyl moieties which may be substituted with one or more of the following: —F, —NO₂, —CN, straight chained or branched C₁-C₇ alkyl, straight chained or branched C₁-C₇ monofluoroalkyl, straight chained or branched C₁-C₇ polyfluoroalkyl, straight chained or branched C₂-C₇ alkenyl, straight chained or branched C₂-C₇ alkynyl, C₃-C₇ cycloalkyl, C₃-C₇ monofluorocycloalkyl, C₃-C₇ polyfluorocycloalkyl, C₅-C_{7 e}cycloalkenyl, —N(R₄)₂, —OR₄, —COR₄, —NCOR₄, —CO₂R₄, —CON(R₄)₂ or (CH₂)ₙ—O—(CH₂)ₘ—CH₃, wherein each m is independently an integer from 0 to 2 inclusive.

As used in the present invention, the term “cycloalkenyl” includes C₅-C₇ cycloalkenyl moieties which may be substituted with one or more of the following: —F, —Cl, —Br, —I, —NO₂, —CN, straight chained or branched C₁-C₇ alkyl, straight chained or branched C₁-C₇ monofluoroalkyl, straight chained or branched C₁-C₇ polyfluoroalkyl, straight chained or branched C₂-C₇ alkenyl, straight chained or branched C₂-C₇ alkynyl, C₃-C₇ cycloalkyl, C₃-C₇ monofluorocycloalkyl, C₃-C₇ polyfluorocycloalkyl, C₅-C₇ cycloalkenyl, —N(R₄)₂, —OR₄, —COR₄, —NCOR₄, —CO₂R₄, —CON(R₄)₂ or (CH₂)ₙ—O—(CH₂)ₘ—CH₃, wherein each m is independently an integer from 0 to 2 inclusive.

In the present invention, the term “heteroaryl” is used to include five and six membered unsaturated rings that may contain one or more oxygen, sulfur, or nitrogen atoms. Examples of heteroaryl groups include, but are not limited to, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, and triazinyl.

In addition the term “heteroaryl” is used to include fused bicyclic ring systems that may contain one or more heteroatoms such as oxygen, sulfur and nitrogen. Examples of such heteroaryl groups include, but are not limited to, indolizinyl, indolyl, isoindolyl, benzo[b]furanyl, benzo[b]thiophenyl, indazolyl, benzimidazolyl, purinyl, benzoxazolyl, benzisoxazolyl, benzo[b]thiazolyl, imidazo[2,1-b]thiazolyl, cinnolinyl, quinazolinyl, quinoxalinyl, 1,8-naphthyridinyl, pteridinyl, quinolinyl, isoquinolinyl, phthalimidyl and 2,1,3-benzothiazolyl.

The term “heteroaryl” also includes those chemical moieties recited above which may be substituted with one or more of the following: —F, —Cl, —Br, —I, —NO₂, —CN, straight chained or branched C₁-C₇ alkyl, straight chained or branched C₁-C₇ monofluoroalkyl, straight chained or branched C₁-C₇ polyfluoroalkyl, straight chained or branched C₂-C₇ alkenyl, straight chained or branched C₂-C₇ alkynyl, C₃-C₇ cycloalkyl, C₃-C₇ monofluorocycloalkyl, C₃-C₇ polyfluorocycloalkyl, C₅-C₇ cycloalkenyl, —N(R₄)₂, —OR₄, —COR₄, —NCOR₄, —CO₂R₄, —CON(R₄)₂ or (CH₂)ₙ—O—(CH₂)ₘ—CH₃, wherein each m is independently an integer from 0 to 2 inclusive.

The term “heteroaryl” further includes the N-oxides of those chemical moieties recited above which include at least one nitrogen atom.

In the present invention the term “aryl” is phenyl or naphthyl. The term “aryl” also includes phenyl and naphthyl which may be substituted with one or more of the following: —F, —Cl, —Br, —I, —NO₂, —CN, straight chained or branched C₁-C₇ alkyl, straight chained or branched C₁-C₇ monofluoroalkyl, straight chained or branched C₁-C₇ polyfluoroalkyl, straight chained or branched C₂-C₇ alkenyl, straight chained or branched C₂-C₇ alkynyl, C₃-C₇ cycloalkyl, C₃-C₇ monofluorocycloalkyl, C₃-C₇ polyfluorocycloalkyl, C₅-C₇ cycloalkenyl, —N(R₄)₂, —OR₄, —SR₄, —OCOR₄, —COR₄, —NCOR₄, —CO₂R₄, —CON(R₄)₂ or (CH₂)ₙ—O—(CH₂)ₘ—CH₃, wherein each m is independently an integer from 0 to 2 inclusive.

The present invention also provides a method of treating a subject in need of treatment for a nerve cellular injury and/or trauma which compromises administering to the subject an effective amount of a galanin-3 receptor antagonist compound, wherein the a galanin-3 receptor antagonist compound has the structure: 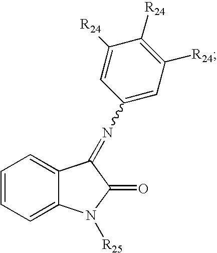
wherein each R₂₄ is independently one or more of the following: H, F, Cl, Br, I, CF₃ or OCH₃;
wherein R₂₅ is methyl, ethyl, allyl or phenyl and the phenyl is optionally substituted with a F, Cl, Br, CF₃, or OR₄; and
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl.
In the methods described herein, the compound contains an imine bond, which can potentially have a Z or E stereoconfiguration. In one embodiment of any of the methods described herein, the compound is a pure Z imine isomer. In one embodiment of any of the methods described herein, the compound is a pure E imine isomer. In one embodiment of any of the methods described herein, the compound is a mixture of Z and E imine isomers.

In the methods described herein, the compound may contain an alkene bond, which can potentially have a Z or E stereoconfiguration. For example, the compound may contain a group Y₂ attached to the 5-position of an indolone ring system, where Y₂ is but-2-en-1-yl. Such a butenyl group can potentially have a Z or E stereoconfiguration. In one embodiment of any of the methods described herein, the compound is a pure Z alkene isomer. In one embodiment of any of the methods described herein, the compound is a pure E alkene isomer. In one embodiment of any of the methods described herein, the compound is a mixture of Z and E alkene isomers.

In the methods described herein, the compound may contain one or more moieties that are capable of chirality. Such moieties may include, but are not limited to, quadrivalent chiral atoms or ring systems with restricted rotation giving rise to perpendicular dissymmetric planes. In one embodiment of any of the methods described herein, the compound is enantiomerically or diastereomerically pure. In one embodiment of any of the methods described herein, the compound is enantiomerically and diastereomerically pure. In one embodiment of any of the methods described herein, the compound is a mixture of enantiomers. In one embodiment of any of the methods described herein, the compound is a mixture of diastereomers.

In one embodiment, the compound is administered orally.

In one embodiment, the compound has the structure: 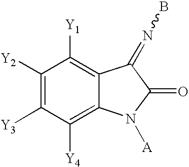
wherein each of Y₁, Y₂, Y₃, and Y₄ is independently —H; straight chained or branched C₁-C₇ alkyl, —CF₃, —F, —Cl, —Br, —I, —OR₄, —N(R₄)₂, or —CON(R₄)₂;
wherein each R₄ is independently —H; straight chained or branched C₁-C₇ alkyl, —CF₃, or phenyl;
wherein A is A′, straight chained or branched C₁-C₇ alkyl, aryl, heteroaryl, aryl(C₁-C₆)alkyl or heteroaryl(C₁-C₆)alkyl; and
wherein A′ is

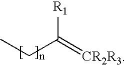

In one embodiment, B is heteroaryl. In another embodiment, B is aryl.

In one embodiment, B is phenyl and the phenyl is optionally substituted with one or more of the following: —H, —F, —Cl, —Br, —CF₃, straight chained or branched C₁-C₇ alkyl, —N(R₄)₂, —OR₄, —COR₄, —NCOR₄, —CO₂R₄, or —CON(R₄)₂.

In one embodiment, A is aryl. In another embodiment, A is heteroaryl.

In one embodiment, the compound is selected from the group consisting of:

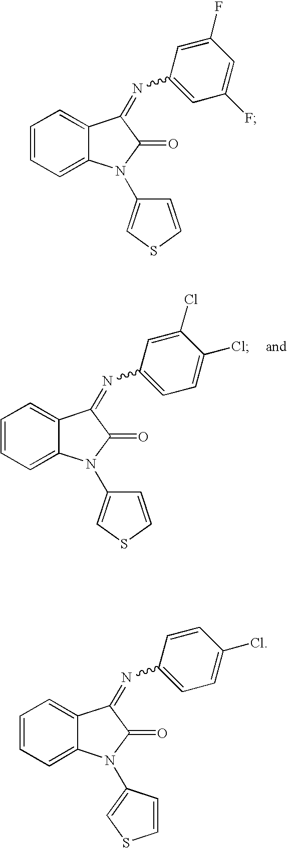

In one embodiment, the compound is selected from the group consisting of:

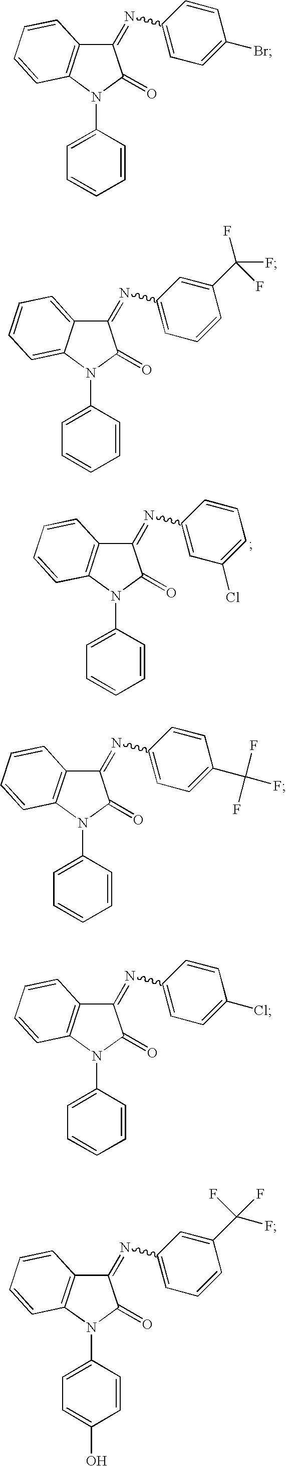

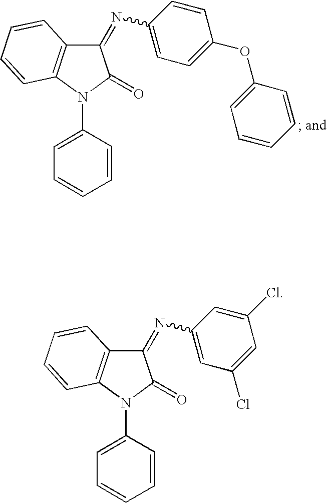

In one embodiment, A is A′ and A′ is 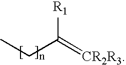
In one embodiment, the compound is:

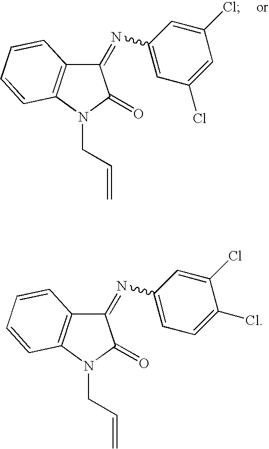

In one embodiment, A is aryl. In another embodiment, B is aryl.

In one embodiment, A is heteroaryl(C₁-C₆)alkyl.

In one embodiment, the compound is:

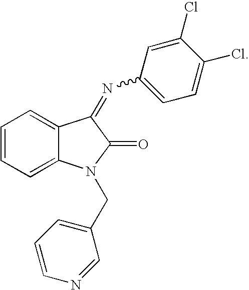

In particular embodiments, the galanin-3 receptor antagonistis is HT-2157 (1,3-dihydro-1-phenyl-3[[3-trifluoromethyl)phenyl]imino]-2H-indol-2-one; CAS No. 303149-14-6.

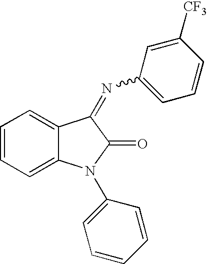

Other examples of Galanin 3 receptor antagonists can be found in, U.S. Pat. No. 7,081,470, U.S. Publication No. US2003/078271A1; and International Publication No. WO2004/093789 which are incorporated by reference in their entirety. The compounds can be prepared using the methodology provided in U.S. Pat. No. 7,081,470, U.S. Publication No. US2003/078271 A1; and International Publication No. WO2004/093789, the teachings of which are incorporated herein by reference.

It is contemplated that the administration of galanin-3 receptor (Gal3R) antagonist compounds can be done alone or with the administration of other compounds for example benzodiazepine or selective serotonin reuptake inhibitors (SSRI).

The method or treatment may comprise administering a combination of primary medications for the condition(s) targeted for treatment and a galanin-3 receptor antagonist. In some cases the galanin-3 receptor antagonist has a synergistic effect with an additional therapeutic agent in treating the disease targeted for treatment. When administered as a combination, the therapeutic compounds can be formulated as separate compositions that are administered at the same time or sequentially at different times or the therapeutic compounds can be given as a single composition.

The mode of administration is preferably at the location of the target cells. In a particular embodiment, the mode of administration is to neurons.

The present invention provides a method for treating inhibiting or ameliorating the effects of injuries or diseases that result in neuronal degeneration or a method for promoting neurogenesis or neurite outgrowth. These methods involve administering to a patient in need thereof an effective amount of at least one galanin-3 receptor antagonist. It has been found the galanin-3 receptor antagonists of the present invention promote neurite outgrowth and neurogenesis.

Alternatively, the at least one galanin-3 receptor antagonist of the present invention is used to treat stem cells or neuronal progenitor cells prior to the cells being administered to the patient by implantation at the site of neuronal degeneration. In some embodiments, methods described herein involve modulating neurogenesis.or neurite outgrowth ex vivo with the galanin-3 receptor antagonist compound such that a composition containing neural stem cells, neural progenitor cells and/or differentiated neural cells can be subsequently administered to an individual to treat a disease or condition. In some embodiments, the method of treatment comprises the steps of contacting a neural stem cell or neural progenitor cell with one or more compounds of the invention to modulate neurite outgrowth and transplanting the cells into a patient in need or treatment. Methods of transplanting stem and progenitor cells are known in the art. In some embodiments, methods described herein allow treatment of diseases or conditions by directly replacing or replenishing damaged or dysfunctional neurons.

The method of the present invention which promotes neurogenesis is involved in cell renewal in the central nervous system (CNS) and includes all types of CNS cells.

An embodiment of the present invention is used to treat primary nervous system injury e.g. closed head injuries and blunt trauma, such as those caused by participation in dangerous sports, penetrating trauma, such as gunshot wounds, hemorrhagic stroke, ischemic stroke, glaucoma, cerebral ischemia, or damages caused by surgery such as tumor excision or may even promote nerve regeneration in order to enhance or accelerate the healing of such injuries or of neurodegenerative diseases such as those discussed below. In addition, the method may be used to treat, inhibit or ameliorate the effects of disease or disorder that results in a degenerative process.

An embodiment of the present invention is used to inhibit secondary degeneration which may otherwise follow primary nervous system injury.

The compounds of the invention may be used to treat various diseases or disorders of the central or peripheral nervous system, including diabetic neuropathy, amyotrophic lateral sclerosis (ALS). Peripheral nerve injuries and peripheral or localized neuropathies including, but not limited to, porphyria, acute sensory neuropathy, chronic ataxic neuropathy, complications of various drugs and toxins, amyloid polyneuropathies, adrenomyeloneuropathy, giant axonal neuropathy may be treated by this method.

In addition the compounds can be used for post-operative treatments such as for tumor removal from CNS and other forms of surgery on the CNS. The compounds can be used for treatment of spinal chord trauma.

The Gal-3 receptor antagonist can be administered together with other components of biologically active agents, such as pharmaceutically acceptable surfactants (e.g., glycerides), excipients (e.g., lactose), stabilizers, preservatives, humectants, emollients, antioxidants, carriers, diluents and vehicles. If desired, certain sweetening, flavoring and/or coloring agents can also be added.

The Gal-3 receptor antagonist can be formulated as a solution, suspension, emulsion or lyophilized powder in association with a pharmaceutically acceptable parenteral vehicle. Examples of such vehicles are water, saline, Ringer's solution, isotonic sodium chloride solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation can be sterilized by commonly used techniques. Suitable pharmaceutical carriers are described in Remington's Pharmaceutical Sciences.

The dosage of Gal-3 receptor antagonist administered to an animal is that amount required to effect a change in neurite outgrowth. The dosage administered to an animal, including frequency of administration, will vary depending upon a variety of factors, including pharmacodynamic characteristics of the particular Gal-3 receptor antagonist, mode and route of administration; size, age, sex, health, body weight and diet of the recipient; nature and extent of symptoms being treated or nature and extent of the cognitive function(s) being enhanced or modulated, kind of concurrent treatment, frequency of treatment, and the effect desired. In the subject application a “therapeutically effective amount” is any amount of a compound which, when administered to a subject suffering from a disease against which the compounds are effective, causes modulation of neurite outgrowth.

The Gal-3 receptor antagonist can be administered in single or divided doses (e.g., a series of doses separated by intervals of days, weeks or months), or in a sustained release form, depending upon factors such as nature and extent of symptoms, kind of concurrent treatment and the effect desired. Other therapeutic regimens or agents can be used in conjunction with the present invention. For example, the Gal-3 receptor antagonist can be administered daily for a period of time.

The present invention will now be illustrated by the following example, which is not to be considered limiting in any way.

### EXPERIMENTAL DETAILS

### Synthesis of Chemical Compounds

The following description illustrates methods that may be used to synthesize the indolone compounds of this invention. The synthesis of the compounds is described in U.S. Ser. No. 11/608,746, filed Dec. 6, 2006, which is incorporated by reference in its entirety.

General Methods

All reactions were performed under an Argon atmosphere and the reagents, neat or in appropriate solvents, were transferred to the reaction vessel via syringe and cannula techniques. Anhydrous solvents were purchased from the Aldrich Chemical Company and used as received.

The compounds described below were named using the ACD/Name Program (version 4.01, Advanced Chemistry Development Inc., Toronto, Ontario, M5H2L3, Canada). The ¹H NMR and ¹³C NMR spectra were recorded at either 300 MHz (GEQE Plus) or 400 MHz (Bruker Avance) in CDCl₃ as solvent and tetramethylsilane as the internal standard unless otherwise noted. Chemical shifts (δ) are expressed in ppm, coupling constants (J) are expressed in Hz, and splitting patterns are described as follows: s=singlet; d=doublet; t=triplet; q=quartet; quintet; sextet; septet; br=broad; m=mutiplet; dd=doublet of doublets; dt=doublet of triplets. Elemental analyses were performed by Robertson Microlit Laboratories, Inc. Unless indicated otherwise, mass spectra were obtained using electrospray ionization (ESI, Micromass Platform II) and MH⁺ is reported. Thin-layer Chromatography (TLC) was carried out on glass plates pre-coated with silica gel 60 F₂₅₄ (0.25 mm, EM Separations Tech.). Preparative TLC was carried out on glass sheets pre-coated with silica gel GF (2 mm, Analtech). Flash column chromatography was performed on Merck silica gel 60 (230-400 mesh). Melting points (mp) were determined in open capillary tubes on a MeI-Temp apparatus and are uncorrected.

The following additional abbreviations are used: HOAc, acetic acid; DIPEA, diisopropylethylamine; DMF, N,N-dimethylformamide; EtOAc, ethyl acetate; MeOH, methanol; TEA, triethylamine; THF, tetrahydrofuran; All solvent ratios are volume/volume unless stated otherwise.

I. General Procedure for Preparing Indolones

The methods that follow demonstrate procedures useful for synthesizing compounds of this invention (illustrated in Schemes 1-5). Substituted isatins useful for synthesizing compounds of this invention can alternatively be obtained using the procedures described in the following references:
Garden, S. J.; Da Silva, L. E.; Pinto, A. C.; Synthetic Communications, 1998, 28, 1679-1689.
Coppola, G. M.; Journal of Heterocyclic Chemistry, 1987, 24, 1249.
Hess, B. A. Jr; Corbino, S.; Journal of Heterocyclic Chemistry, 1971, 8, 161.
Bryant, W. M. III; Huhn, G. F.; Jensen, J. H.; Pierce, M. E.; Stammbach, C.; Synthetic Communications, 1993, 23, 1617-1625.
   General Procedure for Synthesis of Iminoisatins

The appropriately substituted isatin (10 mg-10 g) was placed in a flask and the appropriate aniline (1.0-1.1 equivalents) was added and the mixture was stirred to homogeneity. The mixture was then heated to 110° C. for 2-7 hours and then cooled. Solids were crystallized from hot methanol and filtered, giving the desired products (usually as an inseparable interconverting mixture of E/Z isomers).

Procedure A:

1-(3-THIENYL)-1H-INDOLE-2,3-DIONE: Triethylamine (56.9 mL, 0.408 mol), was added to a mixture of 1H-indole-2,3-dione (15.0 g, 0.102 mol), copper (II) acetate (46.0 g, 0.255 mol), and 3-thienylboronic acid (19.6 g, 0.153 mol) in CH₂Cl₂ (500 mL). The reaction mixture was stirred overnight, filtered through Celite®, rinsed with EtOAc/hexane (1:1, 300 mL), and concentrated in vacuo. The crude product was purified by column chromatography on silica using Hexane/EtOAc (1:1), giving the desired product (1.1 g, 50%).

Procedure B:

(3E)-3-[(4-METHYLPHENYL)IMINO]-1-(3-THIENYL)-1,3-DIHYDRO-2H-INDOL-2-ONE: A solution of 1-(3-thienyl)-1H-indole-2,3-dione (20 mg, 0.087 mmol) in 1% HOAc/MeOH (8 mL) was added to a solution of p-toluidine (19 mg, 0.18 mmol) in 1% HOAc/MeOH (8 mL). The reaction mixture was stirred for 12 h at room temperature, heated at 50° C. for 1 h, and concentrated in vacuo. The residue was purified by preparative TLC on silica using EtOAc/hexanes (3:7, 0.1% TEA) giving the desired product (14 mg, 50%).

Procedure C:

(3Z)-5-BROMO-3-{[3-(TRIFLUOROMETHYL)PHENYL]IMINO}-1,3-DIHYDRO-2H-INDOL-2-ONE: A mixture of 5-bromo-1H-indole-2,3-dione (1.0 g, 0.442 mmol) and 3-trifluoromethylaniline (0.993 g, 6.2 mmol) in a solution of 1% acetic acid in methanol was stirred at 50° C. for 12 h. The crude product was concentrated in vacuo, giving the desired crude product (640 mg, 40%).

Procedure D:

(3Z)-5-BROMO-1-PHENYL-3-{[3-TRIFLUOROMETHYL)PHENYL]IMINO}-1,3-DIHYDRO-2H-INDOL-2-ONE: A mixture of (3Z)-5-bromo-3-{[3-(trifluoromethyl)phenyl]imino}-1,3-dihydro-2H-indol-2-one (100 mg, 0.272 mmol), copper (II) acetate (54 mg, 0.33 mmol), triethylamine (82.8 mg, 0.817 mmol), and benzene boronic acid (40 mg, 0.325 mmol) in 5 mL of CH₂Cl₂ was stirred at room temperature for 12 h. The crude mixture was concentrated in vacuo and purified by preparative TLC using EtOAc:hexane (3:7, 1% triethylamine), giving the desired product (22 mg, 20%).

Procedure E:

(3Z)-1,5-DIPHENYL-3-{[3-(TRIFLUOROMETHYL)PHENYL]IMINO}-1,3-DIHYDRO-2H-INDOL-2-ONE: A mixture of (3Z)-5-bromo-1-phenyl-3-{[3-(trifluoromethyl)phenyl]imino}-1,3-dihydro-2H-indol-2-one (22 mg, 0.05 mmol), tetrakis(triphenylphosphine)palladium(0) (12.0 mg, 0.01 mmol), benzene boronic acid (10 mg, 0.08 mmol) in THF (5 mL), and aqueous Na₂CO₃ (2M, 100 μL) was heated at 67° C. for 24 h. The crude product was concentrated in vacuo and the residue was extracted with CH₂Cl₂ (3×1 ml), concentrated, and purified by preparative TLC using 10% methanol in CHCl₃, giving the desired product (4 mg, 18%).
   Procedure F:

1-[(5-CHLORO-1-BENZOTHIEN-3-YL)METHYL]-2H-INDOLE-2,3-DIONE: A solution of isatin (125 mg, 0.85 mmol) in anhydrous dioxane (10 mL) was added dropwise to a solution of sodium hydride (60% dispersion in mineral oil, 25 mg, 0.62 mmol) in anhydrous dioxane (10 mL) at 0° C. under argon. The mixture was allowed to stir for 5 minutes and then a solution of 3-(bromomethyl)-5-chlorobenzo[b]thiophene (267 mg, 1.02 mmol) in dioxane (10 mL) was added dropwise to the reaction mixture. The reaction mixture was heated at reflux under argon for 16 h and concentrated in vacuo. The crude material was purified by preparative TLC using 1:24 methanol in chloroform as the eluent, giving the desired product as a yellow solid (125 mg, 0.38 mmol, 45%).

Procedure G:

1-[(5-CHLORO-1-BENZOTHIEN-3-YL)METHYL]-3-{[3-(TRIFLUOROMETHYL)PHENYL]IMINO}-1,3-DIHYDRO-2H-INDOL-2-ONE: A mixture of 1-[(5-chloro-1-benzothien-3-yl)methyl]-2H-indole-2,3-dione (50 mg, 0.15 mmol) and 3-trifluoromethylaniline (0.020 mL, 0.15 mmol) was heated neat at 140° C. for 2 h. The crude material was purified by preparative TLC using a mixture of 1:3 ethyl acetate and hexane as the eluent giving the desired product as a yellow solid (13 mg, 0.030 mmol, 18%).

Procedure H:

6-METHOXY-1-PHENYL-1H-INDOLE-2,3-DIONE: A solution of N-(3-methoxyphenyl)-N-phenylamine (1.14 g, 5.72 in ether (3 mL) was added to a solution of oxalyl chloride (728 g, 5.75 mmol) and heated at reflux for 1 h. The resulting mixture was cooled to room temperature, concentrated to dryness, and redissolved in nitrobenzene (35 mL). The solution was added to a solution of AlCl₃ in nitrobenzene (0.762 g, 5.72 mmol), and the resulting mixture was heated at 70° C. for 16 h. The crude product was concentrated in vacuo and purified by column chromatography using EtOAc/hexane (1:1), giving the desired product 60, mg, 50%). Compounds 2-17, inclusive, were purchased from Bionet Research Ltd., 3 Highfield Industrial Estate, Camelford, Cornwall PL32 9QZ, UK. These compounds can also be synthesized using the General Procedure described above.

Compound 1: 3-[(2-METHOXYPHENYL)IMINO]-1-PHENYL-1,3-DIHYDRO-2H-INDOL-2-ONE

Compound 2: 1-PHENYL-3-[[3-(TRIFLUOROMETHYL)PHENYL]IMINO]-1,3-DIHYDRO-2H-INDOL-2-ONE

Compound 3: 3-[(3-METHYLPHENYL)IMINO]-1-PHENYL-1,3-DIHYDRO-2H-INDOL-2-ONE

Compound 4: 3-[(3-CHLOROPHENYL)IMINO]-1-PHENYL-1,3-DIHYDRO-2H-INDOL-2-ONE

Compound 5: 1-PHENYL-3-[[4-(TRIFLUOROMETHYL)PHENYL]IMINO]-1,3-DIHYDRO-2H-INDOL-2-ONE

Compound 6: 3-[(4-METHYLPHENYL)IMINO]-1-PHENYL-1,3-DIHYDRO-2H-INDOL-2-ONE

Compound 7: 3-[(4-CHLOROPHENYL)IMINO]-1-PHENYL-1,3-DIHYDRO-2H-INDOL-2-ONE

Compound 8: 3-[(4-BROMOPHENYL)IMINO]-1-PHENYL-1,3-DIHYDRO-2H-INDOL-2-ONE

Compound 9: 3-[(4-FLUOROPHENYL)IMINO]-1-PHENYL-1,3-DIHYDRO-2H-INDOL-2-ONE

Compound 10: 3-[(4-PHENOXYPHENYL)IMINO]-1-PHENYL-1,3-DIHYDRO-2H-INDOL-2-ONE

Compound 11: 3-[(4-ETHOXYPHENYL)IMINO]-1-PHENYL-1,3-DIHYDRO-2H-INDOL-2-ONE

Compound 12: 3-[(4-METHOXYPHENYL)IMINO]-1-PHENYL-1,3-DIHYDRO-2H-INDOL-2-ONE

Compound 13: 3-[(3,5-DICHLOROPHENYL)IMINO]-1-PHENYL-1,3-DIHYDRO-2H-INDOL-2-ONE

Compound 14: 3-[(3,5-DIMETHYLPHENYL)IMINO]-1-PHENYL-1,3-DIHYDRO-2H-INDOL-2-ONE

Compound 15: 1-ALLYL-3-[(3,4-DICHLOROPHENYL)IMINO]-1,3-DIHYDRO-2H-INDOL-2-ONE

Compound 16: 1-ALLYL-3-[(3,5-DICHLOROPHENYL)IMINO]-1,3-DIHYDRO-2H-INDOL-2-ONE

Compound 17: 3-[(4-BROMOPHENYL)IMINO]-1-ISOPROPYL-1,3-DIHYDRO-2H-INDOL-2-ONE

Compound 18: 1-[(5-CHLORO-2-THIENYL)METHYL]-3-{[3-(TRIFLUOROMETHYL)PHENYL]IMINO}1,3-DIHYDRO-2H-INDOL-2-ONE: A mixture of 1-[(5-chloro-2-thienyl)methyl]-2H-indole-2,3-dione (25 mg, 0.09 mmol) (prepared as described below) and 3-trifluoromethylaniline (11.3 μL, 0.09 mmol) was heated neat at 140° C. for 2 h. The crude material was purified by preparative TLC using a mixture of 3:7 ethyl acetate in hexane as the eluent, giving the desired product (23 mg, 0.05 mmol, 61%). ¹H NMR (400 MHz): δ (major isomer) 7.57 (t, J=7.7, 1H), 7.53 (t, J=7.8, 1H), 7.33 (t, J=7.8, 1H), 7.28 (s, 1H), 7.19 (d, J=7.6, 2H), 6.94-6.72 (m, 4H), 6.56 (d, J=7.7, 1H), 5.02 (s, 2H); ESI-MS m/z found 421 (MH⁺).

1-[(5-CHLORO-2-THIENYL)METHYL]-2H-INDOLE-2,3-DIONE: A solution of isatin (125 mg, 0.85 mmol) in anhydrous dioxane (10 mL) was added dropwise to a solution of sodium hydride (60% dispersion in mineral oil, 24 mg, 0.62 mmol) in anhydrous dioxane (10 mL) at 0° C. under argon. The mixture was allowed to stir for 5 minutes and then 2-chloro-5-(chloromethyl)thiophene (0.12 mL, 1.02 mmol) in dioxane (10 mL) was added dropwise to the resulting mixture. The reaction mixture was heated at reflux under argon for 16 h and concentrated in vacuo. The crude material was purified by preparative TLC using 1:24 methanol in chloroform as the eluent, giving the desired product as a yellow solid (53 mg, 0.19 mmol, 22%). ¹H NMR (400 MHz): δ7.62 (d, J=7.4, 1H), 7.56 (t, J=7.8, 1H), 7.14 (t, J=7.7, 1H), 6.94 (d, J=8.0, 1H), 6.90 (d, J=3.2, 1H), 6.78 (d, J=3.7, 1H), 4.90 (s, 2H).

Compound 19: 1-(3-THIENYL)-3-{[3-(TRIFLUOROMETHYL)PHENYL]IMINO}-1,3-DIHYDRO-2H-INDOL-2-ONE: A mixture of 1-(3-thienyl)-2H-indole-2,3-dione (25 mg, 0.11 mmol) (prepared as described below) and 3-trifluoromethylaniline (14 uL, 0.11 mmol) was heated neat at 140° C. for 2 h. The crude material was purified by preparative TLC using a mixture of 3:7 ethyl acetate and hexane as the eluent, giving the desired product as a yellow solid (7.3 mg, 0.02 mmol, 22%). ¹H NMR (400 MHz) δ7.62-7.19 (m, 9H), 6.94 (d, J=8.0, 1H), 6.76 (t, J=7.6, 1H); ESI-MS m/z found 373 (MH⁺).

1-(3-THIENYL)-2H-INDOLE-2,3-DIONE: Copper(II) acetate monohydrate (4.25 g, 23.4 mmol) was heated at reflux in acetic anhydride (30 mL) for 2 h. The mixture was filtered and washed with anhydrous ether (500 mL). The solid was dried in vacuo at 55° C. for 16 h. Dichloromethane (1 mL) was added to a mixture of copper(II) acetate (62 mg, 0.34 mmol), isatin (50 mg, 0.34 mmol), and thiophene-3-boronic acid (87 mg, 0.68 mmol), followed by triethylamine (0.10 mL, 0.68 mmol) under argon. The resulting solution was stirred for 16 h at room temperature. The reaction mixture was then recharged with 0.10 mmol copper(II) acetate, 0.10 mmol of 3-thiophene boronic acid, and 1 drop of triethylamine, and the mixture was heated at 50° C. for 6 h. The crude material was purified by preparative TLC using 3:97 methanol in chloroform as the eluent, giving the desired product as a yellow solid (25 mg, 0.11 mmol, 33%). ¹H NMR (400 MHz): δ 7.70 (d, J=7.5, 1H), 7.58 (t, J=7.8, 1H), 7.50 (d, J=5.1, 1H), 7.48 (s, 1H), 7.24 (d, J=5.1, 1H), 7.18 (t, J=7.51, 1H), 7.05 (d, J=8.0, 1H).

Compound 20: 2-METHYL-5-[(2-OXO-1-PHENYL-1,2-DIHYDRO-3H-INDOL-3-YLIDENE)AMINO]-2H-ISOINDOLE-1,3(2H)-DIONE: A mixture of 1-phenylisatin (50 mg, 0.22 mmol) and 4-amino-N-methylpthalimide (40 mg, 0.22 mmol) was heated neat at 215° C. for 2 h. The crude material was purified by preparative TLC using a mixture of 3:7 ethyl acetate and hexane as the eluent, giving the desired product as a yellow solid (8 mg, 0.02 mmol, 10%). ¹H NMR (400 MHz): δ 7.88 (d, J=7.8, 1H), 7.83-7.80 (m, 1H), 7.51 (t, J=7.5, 1H), 7.47-7.18 (m, 6H), 7.02 (t, J=8.0, 1H), 6.91-6.79 (m, 2H), 6.58 (d, J=7.5, 1H), 3.22 (s, 3H); ESI-MS m/z found 382 (MH⁺).

Compound 21: 1-[(5-CHLORO-1-BENZOTHIEN-3-YL)METHYL]-3-{[3-(TRIFLUOROMETHYL)PHENYL]IMINO}-1,3-DIHYDRO-2H-INDOL-2-ONE: 1-[(5-CHLORO-1-BENZOTHIEN-3-YL)METHYL]-2H-INDOLE-2,3-DIONE was prepared by Procedure F. ¹H NMR (400 MHz): δ 7.89 (s, 1H), 7.79 (d, J=8.5, 1H), 7.65 (d, J=7.5, 1H), 7.54 (t, J=8.0, 1H), 7.42 (s, 1H), 7.38 (d, J=8.5, 1H), 7.14 (t, J=7.5, 1H), 6.88 (d, J=7.8, 1H), 5.13 (s, 2H). From this intermediate, 1-[(5-CHLORO-1-BENZOTHIEN-3-YL)METHYL]-3-{[3-(TRIFLUOROMETHYL)PHENYL]-IMINO}-1,3-DIHYDRO-2H-INDOL-2-ONE was prepared by Procedure G. ¹H NMR (400 MHz): δ 7.98 (d, J=2.0, 1H), 7.80 (d, J=8.6, 1H), 7.58 (t, J=7.7, 1H), 7.52 (d, J=8.1, 1H), 7.43 (s, 1H), 7.38 (dd, J=8.6, 1.9, 1H), 7.31 (overlapping singlet and dt, J=1.2, 7.8, 2H), 7.24 (d, J=7.8, 1H), 6.87 (d, J=7.9, 1H), 6.77 (t, J=7.7, 1H), 6.59 (d, J=7.7, 1H), 5.20 (s, 2H). ESI-MS m/z found 471 (MH⁺ with ³⁵Cl), 473 (MH⁺ with ³⁷⁵Cl).

Compound 22: 3-(1H-INDOL-5-YLIMINO)-1-PHENYL-1,3-DIHYDRO-2H-INDOL-2-ONE: 1-Phenylisatin (51.8 mg, 0.23 mmol) and 5-aminoindole (31 mg, 0.23 mmol) were mixed and heated at 140° C. for 2 h. The resulting crude product was purified by preparative TLC using ethyl acetate/hexane (6:4) as the eluent, giving the desired product as a yellow solid (10.8 mg, 14%). ¹H NMR (400 MHz): δ 8.28 (s, 1H), 7.57 (t, J=7.7, 2H), 7.49-7.40 (m, 6H), 7.29-7.23 (m, 1H), 7.03 (dd, J=8.5, 1.7, 1H), 6.98 (d, J=7.6, 1H), 6.83 (d, J=8.0, 1H), 6.74, J=7.6, 1H), 6.59 (s, 1H); ESI-MS m/z found 338 (MH⁺).

Compound 23: 3-[(6-CHLORO-3-PYRIDINYL)IMINO]-1-PHENYL-1,3-DIHYDRO-2H-INDOL-2-ONE: 1-Phenylisatin (23.0 mg, 0.10 mmol) and 5-amino-2-chloropyridine (12.8 mg, 0.10 mmol) were mixed and heated at 140° C. for 7 h. The resulting crude product was purified by preparative TLC using hexane/ethyl acetate (8:2) as the eluent, giving the desired product as a yellow solid (19.7 mg, 59%). ¹H NMR (400 MHz) δ 8.15 (d, J=8, 1H), 7.6-7.2 (m, 9H), 6.85-6.75 (m, 2H); ESI-MS m/z found 334 (MH⁺).

Compound 24:3-[(2-METHYL-1,3-BENZOTHIAZOL-5-YL)IMINO]-1-PHENYL-1,3-DIHYDRO-2H-INDOL-2-ONE: 5-Amino-2-methylbenzothiazole (52.2 mg, 0.31 mmol) was mixed with 1-phenylisatin (69.7 mg, 0.31 mmol) and heated at 140° C. for 3 h. The resulting crude product was purified by preparative TLC using ethyl acetate/hexane (6:4) as the eluent to give the desired product as a yellow solid (36.9 mg, 32.3%). ¹H NMR: δ 7.9-6.7 (m, 12H), 2.9 (s, 3H). ESI-MS m/z found 370 (MH⁺).

Compound 25: (3Z)-3-[(3,4-DICHLOROPHENYL)IMINO]-1-(2-PYRIDINYLMETHYL)-1,3-DIHYDRO-2H-INDOL-2-ONE: Prepared by Procedures F (for substitution of 2-picolyl chloride) and G. ¹H NMR (400 MHz, CDCl₃) δ 8.51-8.46 (m, 1H), 7.87-7.78 (m, 1H), 7.64 (d, 1H, J=7.1), 7.53-7.31 (m, 5H), 7.28 (d, 1H, J=4.1), 7.12 (d, 1H, J=8.1), 6.58-6.53 (m, 1H), 5.51 (s, 2H); ESI-MS m/z 381 (MH⁺).

Compound 26: (3Z)-3-[(3,4-DICHLOROPHENYL)IMINO]-1-[(3,5-DIMETHYL-4-ISOXAZOLYL)METHYL]-1,3-DIHYDRO-2H-INDOL-2-ONE: Prepared by Procedures F (for substitution of 4-chloromethyl-3,5-dimethylisoxazole) and B (microwave heating). ¹H NMR (400 MHz, CDCl₃) δ 7.63 (d, 1H, J=9.1), 7.46 (dt, 1H, J=8.1, 2.0), 7.28 (d, 1H, J=2.1), 7.02 (d, 1H, J=2.0), 6.88 (dt, 1H, J 8.0, 2.1), 6.74-6.72 (m, 1H), 6.72-6.70 (m, 1H), 5.53 (s, 2H), 2.50 (s, 3H), 2.24 (s, 3H); ESI-MS m/z 399 (MH⁺).

Compound 27: (3Z)-3-[(3,4-DICHLOROPHENYL)IMINO]-1-[3-(TRIFLUOROMETHYL)PHENYL]-1,3-DIHYDRO-2H-INDOL-2-ONE: Prepared by

Procedures A and B. ¹H NMR (400 MHz, CDCl₃) δ 7.90-7.87 (m, 1H), 7.83-7.79 (m, 1H), 7.67 (d, 1H, J=8), 7.46-7.40 (m, 1H), 7.33 (d, 1H, J=2), 7.08-7.05 (m, 1H), 6.96-6.80 (m, 5H); ESI-MS m/z 435 (MH⁺).

Compound 28: (3Z)-1-(3,5-DICHLOROPHENYL)-3-[(3,4-DICHLOROPHENYL)IMINO]-1,3-DIHYDRO-2H-INDOL-2-ONE: Prepared by Procedures A and B. ¹H NMR (400 MHz, CDCl₃) δ 7.93 (d, 1H, J=8.1), 7.79 (d, 1H, J=6.0), 7.72-7.68 (m, 1H), 7.59-7.45 (m, 1H), 7.46 (d, 1H, J=8.1), 7.32 (dt, 1H, J=8.0, 2.1), 7.23 (d, 1H, J=2.5), 6.97 (dd, 1H, J=8.0, 2.1), 6.92-6.87 (m, 1H), 6.85-6.81 (m, 1H); ESI-MS m/z 435 (MH⁺).

Compound 29: (3Z)-3-[(3,4-DICHLOROPHENYL)IMINO]-6-METHOXY-1-PHENYL-1,3-DIHYDRO-2H-INDOL-2-ONE: Prepared by Procedures H and B. ¹H NMR (400 MHz, CDCl₃) δ 7.69-7.54 (m, 1H), 7.53-7.38 (m, 3H), 7.29 (d, 1H, J=2.0), 7.17 (d, 1H, J=8.1), 7.12 (d, 1H, J=8.0), 6.84 (d, 1H, J=2.5), 6.78 (d, 1H, J=8), 6.6 (dd, 2H, J=8.0, 2.0), 6.55 (dd, 2H, J=8.1, 2.5); ESI-MS m/z (398 MH⁺).

Compound 30: (3Z)-3-[(4-CHLORO-3-METHYLPHENYL)IMINO]-1-(3-THIENYL)-1,3-DIHYDRO-2H-INDOL-2-ONE: Prepared by Procedures A and B (80° C.). ¹H NMR (400 MHz, CDCl₃) δ 7.69-7.62 (m, 2H), 7.49 (s, 1H), 7.47 (s, 1H), 7.41 (dt, 1H, J=7.1, 1.6), 7.3 (dd, 1H, J=5.0, 1.6), 7.05-6.97 (m, 1H, 6.93-6.86 (m, 1H), 6.77 (m, 1H), 6.56 (m, 1H), 2.53 (s, 3H); ESI-MS m/z 353 (MH⁺).

Compound 31: (3Z)-3-(2-NAPHTHYLIMINO)-1-(3-THIENYL)-1,3-DIHYDRO-2H-INDOL-2-ONE: Prepared by Procedures A and B (80° C.). ¹H NMR (400 MHz, CDCl₃) δ 8.15 (d, 1H, J=9.1), 8.06-7.99 (m, 1H), 7.89-7.80 (m, 1H), 7.78-7.71 (m, 1H), 7.71-7.47 (m, 4H), 7.41-7.35 (m, 1H), 7.33 (d, 1H, J=5.2), 7.28 (d, 1H, J=6.8.1), 7.00 (d, 1H, J=8.0), 6.76 (t, 1H, J=7.8), 6.67 (d, 1H, J=7.9); ESI-MS m/z 355 (MH⁺).

Compound 32: (3Z)-3-[(4-CHLOROPHENYL)IMINO]-1-(3-THIENYL)-1,3-DIHYDRO-2H-INDOL-2-ONE: Prepared by Procedures A and B (80° C.). ¹H NMR (400 MHz, CDCl₃) δ 7.69-7.56 (m, 2H), 7.54-7.48 (m, 1H), 7.41 (dt, 1H, J=8, 2), 7.32-7.28 (m, 1H), 7.11-6.99 (m, 3H)h , 6.89 (dt, 1H, J=8), 6.77-6.73 (m, 1H), 6.66-6.33 (m, 1H); ESI-MS m/z 339 (MH⁺).

Compound 33: (3Z)-3-[(4-IODOPHENYL)IMINO]-1-(3-THIENYL)-1,3-DIHYDRO-2H-INDOL-2-ONE: Prepared by Procedures A and B (1% HOAc in MeOH). ¹H NMR (400 MHz, CDCl₃) δ 7.79-7.74 (m, 2H), 7.53-7.48 (m, 2H), 7.35 (dt, 1H, J=8.0, 1.2), 7.29-7.24 (m, 1H), 6.98 (d, 1H, J=8.0), 6.89-6.75 (m, 4H); ESI-MS m/z 431 (MH⁺).

Compound 34: (3Z)-3-[(4-METHYLPHENYL)IMINO]-1-(3-THIENYL)-1,3-DIHYDRO-2H-INDOL-2-ONE: Prepared by Procedures A and B (1% HOAc in MeOH). ¹H NMR (400 MHz, CDCl₃) δ 7.52-7.44 (m, 2H), 7.35-7.22 (m, 4H), 6.99-6.93 (m, 3H), 6.87-6.78 (m, 2H), 2.42 (s, 3H); ESI-MS m/z 319 (MH⁺).

Compound 35: (3Z)-3-[(3,5-DIFLUOROPHENYL)IMINO]-1-(3-THIENYL)-1,3-DIHYDRO-2H-INDOL-2-ONE: Prepared by Procedures A and B (1% HOAc in MeOH). ¹H NMR (400 MHz, CDCl₃) δ 7.54-7.16 (m, 4H), 6.99 (dt, 1H, J=8.2, 0.8), 6.89 (dt, 1H, J=7.7, 1.1), 6.76 (d, 1H, J=7.5), 6.71 (tt, 1H, J=9.3, 2.3), 6.64-6.57 (m, 2H); ESI-MS m/z 341 (MH⁺).

Compound 36: ETHYL 3-{[(3Z)-2-OXO-1-(3-THIENYL)-1,2-DIHYDRO-3H-INDOL-3-YLIDENE]AMINO}BENZOATE: Prepared by Procedures A and B (1% HOAc in MeOH). ¹H NMR (400 MHz, CDCl₃) δ 7.96 (d, 1H, J=7.4), 7.75-7.17 (m, 6H), 6.98 (d, 1H, J=8.0), 6.87-6.78 (m, 2H), 6.63 (d, 1H, J=7.8), 4.45-4.32 (m, 2H), 1.43-1.33 (m, 3H); ESI-MS m/z 377 (MH+).

Compound 37: (3Z)-3-[(6-CHLORO-3-PYRIDINYL)IMINO]-1-(3-THIENYL)-1,3-DIHYDRO-2H-INDOL-2-ONE: Prepared by Procedures A and B (1% HOAc in MeOH). ¹H NMR (400 MHz, CDCl₃) δ 8.21-6.81 (m, 10H); ESI-MS m/z 340 (MH⁺).

Compound 38: 3Z)-3-[(4-PHENOXYPHENYL)IMINO]-1-(3-THIENYL)-1,3-DIHYDRO-2H-INDOL-2-ONE: Prepared by Procedures A and B (1% HOAc in MeOH). ¹H NMR (400 MHz, CDCl₃) δ 7.85-6.70 (m, 16H); ESI-MS m/z 397 (MH⁺).

Compound 39: (3Z)-3-[(4-BROMOPHENYL)IMINO]-1-(3-THIENYL)-1,3-DIHYDRO-2H-INDOL-2-ONE: Prepared by Procedures A and G. ¹H NMR (400 MHz, CDCl₃) δ 7.82-6.55 (m, 11H); ESI-MS m/z 383 (MH⁺).

Compound 40: (3Z)-3-[(3-CHLOROPHENYL)IMINO]-1-(3-THIENYL)-1,3-DIHYDRO-2H-INDOL-2-ONE: Prepared by Procedures A and G. ¹H NMR (400 MHz, CDCl₃) δ 7.55-6.50 (m, 11H); ESI-MS m/z 339 (MH⁺).

Compound 41: (3Z)-3-[(3-METHYLPHENYL)IMINO]-1-(3-THIENYL)-1,3-DIHYDRO-2H-INDOL-2-ONE: Prepared by Procedures A and B (1% HOAc in MeOH). ¹H NMR (400 MHz, CDCl₃) δ 7.67-6.78 (m, 11H), 2.39 (s, 3H); ESI-MS m/z 319 (MH⁺).

Compound 42: (3Z)-3-[(3,4-DICHLOROPHENYL)IMINO]-1-(3-THIENYL)-1,3-DIHYDRO-2H-INDOL-2-ONE: Prepared by Procedures A and B (1% HOAc in MeOH). ¹H NMR (400 MHz, CDCl₃) δ 7.82-6.80 (m, 10H); ESI-MS m/z 373 (MH⁺).

Compound 43: (3Z)-1-(2-PYRIDINYLMETHYL)-3-{[3-(TRIFLUOROMETHYL)PHENYL]IMINO}-1,3-DIHYDRO-2H-INDOL-2-ONE: Prepared by Procedure B. ESI-MS m/z 382 (MH⁺).

Compound 44: (3Z)-3-[(3,5-DICHLOROPHENYL)IMINO]-1-(2-PYRIDINYLMETHYL)-1,3-DIHYDRO-2H-INDOL-2-ONE: Prepared by Procedure B. ESI-MS m/z 382 (MH⁺).

Compound 45: (3Z)-1-[(3,5-DIMETHYL-4-ISOXAZOLYL)METHYL]-3-{[3-(TRIFLUOROMETHYL)PHENYL]IMINO}-1,3-DIHYDRO-2H-INDOL-2-ONE: Prepared by Procedure B. ESI-MS m/z 400 (MH⁺).

Compound 46: (3Z)-3-[(3,4-DIFLUOROPHENYL)IMINO]-1-(3-PYRIDINYLMETHYL)-1,3-DIHYDRO-2H-INDOL-2-ONE: Prepared by Procedures F (for substitution of 3-picolylchloride) and B. ESI-MS m/z 350 (MH⁺).

Compound 47: (3Z)-1-(3-PYRIDINYLMETHYL)-3-{[3-(TRIFLUOROMETHYL)PHENYL]IMINO}-1,3-DIHYDRO-2H-INDOL-2-ONE: Prepared by Procedure B. ESI-MS m/z 382 ((MH⁺).

Compound 48: (3Z)-3-[(3,4-DIFLUOROPHENYL)IMINO]-1-(2-PYRIDINYLMETHYL)-1,3-DIHYDRO-2H-INDOL-2-ONE: Prepared by Procedure B. ESI-MS m/z 350 (MH⁺).

Compound 49: (3Z)-3-[(3,5-DICHLOROPHENYL)IMINO]-1-(3-PYRIDINYLMETHYL)-1,3-DIHYDRO-2H-INDOL-2-ONE: Prepared by Procedure B. ESI-MS m/z 384 (MH⁺).

Compound 50: (3Z)-3-[(3,5-DICHLOROPHENYL)IMINO]-1-[(3,5-DIMETHYL-4-ISOXAZOLYL)METHYL]-1,3-DIHYDRO-2H-INDOL-2-ONE: Prepared by Procedure B. ESI-MS m/z 402 (MH⁺).

Compound 51: (3Z)-1-PHENYL-3-(5-QUINOLINYLIMINO)-1,3-DIHYDRO-2H-INDOL-2-ONE: Prepared by Procedure G. ¹H NMR (400 MHz, CDCl₃) δ 9.38-9.32 (m, 1H), 8.55-8.50 (m, 1H), 8.01-6.62 (m, 12H), 6.43-6.35 (m, 1H); ESI-MS m/z 350 (MH⁺).

Compound 52: (3Z)-3-[(4-IODOPHENYL)IMINO]-1-PHENYL-1,3-DIHYDRO-2H-INDOL-2-ONE: Prepared by Procedure B (0.1% HOAc, 80° C., 92 h, 4 eq RNH₂, 3 Å molecular sieves). ESI-MS m/z 425 (MH⁺).

Compound 53: (3Z)-3-[(3,4-DIFLUOROPHENYL)IMINO]-1-PHENYL-1,3-DIHYDRO-2H-INDOL-2-ONE: Prepared by Procedure B (0.1% HOAc, 80° C., 92 h, 4 eq RNH₂, 3 Å molecular sieves). ESI-MS m/z 335 (MH⁺).

Compound 54: (3Z)-3-[(2-CHLORO-4-METHYLPHENYL)IMINO]-1-PHENYL-1,3-DIHYDRO-2H-INDOL-2-ONE: Prepared by Procedure B (0.1% HOAc, 80° C., 92 h, 4 eq RNH₂, 3 Å molecular sieves). ESI-MS m/z 347 (MH⁺ with ³⁷Cl), 349 (MH⁺ with ³⁷Cl).

Compound 55: (3Z)-3-[(2,4-DIMETHOXYPHENYL)IMINO]-1-PHENYL-1,3-DIHYDRO-2H-INDOL-2-ONE: Prepared by Procedure B (0.1% HOAc, 80° C., 92 h, 4 eq RNH₂, 3 Å molecular sieves). ESI-MS m/z 359 (MH⁺).

Compound 56: 3-{[(3Z)-2-OXO-1-PHENYL-1,2-DIHYDRO-3H-INDOL-3-YLIDENE]AMINO}BENZONITRILE: Prepared by Procedure B (0.1% HOAc, 80° C., 92 h, 4 eq RNH₂, 3 Å molecular sieves). ESI-MS m/z 324 (MH⁺).

Compound 57: (3Z)-3-{[2-METHYL-5-(TRIFLUOROMETHYL)PHENYL]IMINO}-1-PHENYL-1,3-DIHYDRO-2H-INDOL-2-ONE: Prepared by Procedure B (0.1% HOAc, 80° C., 92 h, 4 eq RNH₂, 3 Å molecular sieves). ESI-MS m/z 381 (MH⁺).

Compound 58: (3Z)-3-[(4-CHLORO-3-METHYLPHENYL)IMINO]-1-(3-THIENYL)-1,3-DIHYDRO-2H-INDOL-2-ONE: Prepared by Procedures A and B (80° C.). ESI-MS m/z 353 (MH⁺).

Compound 59: (3Z)-3-(6-QUINOLINYLIMINO)-1-(3-THIENYL)-1,3-DIHYDRO-2H-INDOL-2-ONE: Prepared by Procedures A and B (80° C.). ESI-MS m/z 356 (MH⁺).

Compound 60: (3Z)-3-[(4-CHLOROPHENYL)IMINO]-1-(3-THIENYL)-1,3-DIHYDRO-2H-INDOL-2-ONE: Prepared by Procedures A and B (80° C.). ESI-MS m/z 339 (MH⁺).

Compound 61: (3Z)-3-[(3-ISOPROPYLPHENYL)IMINO]-1-(3-THIENYL)-1,3-DIHYDRO-2H-INDOL-2-ONE: Prepared by Procedures A and B (80° C.). ESI-MS m/z 347 (MH⁺).

Compound 62: (3Z)-3-[(4-CYCLOHEXYLPHENYL)IMINO]-1-(3-THIENYL)-1,3-DIHYDRO-2H-INDOL-2-ONE: Prepared by Procedures A and B (80° C.). ESI-MS m/z 387 (MH⁺).

Compound 63: (3Z)-3-(1,3-BENZOTHIAZOL-6-YLIMINO)-1-PHENYL-1,3-DIHYDRO-2H-INDOL-2-ONE: Prepared by Procedure G. ESI-MS m/z 356 (MH⁺).

Compound 64: (3Z)-3-(1H-INDAZOL-6-YLIMINO)-1-PHENYL-1,3-DIHYDRO-2H-INDOL-2-ONE: Prepared by Procedure G. ESI-MS m/z 339 (MH⁺).

Compound 65: (3Z)-3-[(3-CHLOROPHENYL)IMINO]-6-METHOXY-1-PHENYL-1,3-DIHYDRO-2H-INDOL-2-ONE: Prepared by Procedures H and G. ESI-MS m/z 363 (MH⁺).

Compound 66: (3Z)-6-METHOXY-1-PHENYL-3-{[3-(TRIFLUOROMETHYL)PHENYL]IMINO}-1,3-DIHYDRO-2H-INDOL-2-ONE: Prepared by Procedures H and G. ESI-MS m/z 397 (MH⁺).

Compound 67: (3Z)-3-[(3-BROMOPHENYL)IMINO]-1-PHENYL-1,3-DIHYDRO-2H-INDOL-2-ONE: Prepared by Procedure B. ESI-MS m/z 378 (MH⁺).

Compound 68: (3Z)-1,5-DIPHENYL-3-{[3-(TRIFLUOROMETHYL)PHENYL]IMINO}-1,3-DIHYDRO-2H-INDOL-2-ONE: Prepared by Procedures C, D, and E. ESI-MS m/z 443 (MH⁺).

Compound 69: (3Z)-1-(4-HYDROXYPHENYL)-3-{[3-(TRIFLUOROMETHYL)PHENYL]IMINO}-1,3-DIHYDRO-2H-INDOL-2-ONE: Prepared by Procedures G (6 eq of aniline) and D. ESI-MS m/z 383 (MH⁺).

Compound 70: (3Z)-3-[(3,4-DICHLOROPHENYL)IMINO]-1-(3-PYRIDINYLMETHYL)-1,3-DIHYDRO-2H-INDOL-2-ONE: Prepared by Procedure G (75° C., 2 h). ESI-MS m/z 383 (MH⁺).

Compounds 1-70 as described above are merely illustrative of indolone compounds which may be utilized in the methods of the present invention. Further indolone compounds may be obtained utilizing the methods shown in Schemes 1-5 and procedures generally known in the art.

It may be necessary to incorporate protection and deprotection strategies for substituents such as amino, amido, carboxylic acid, and hydroxyl groups in the synthetic methods described above to form indolone derivatives. Methods for protection and deprotection of such groups are in well-known in the art, and may be found, for example in Green, T. W. and Wuts, P. G. M. (1991) Protection Groups in Organic Synthesis, 2nd Edition John Wiley & Sons, New York.

The structures of Compounds 1-70 are illustrated in Tables 1 and 1a.

TABLE 1
Chemical Structures of Compounds

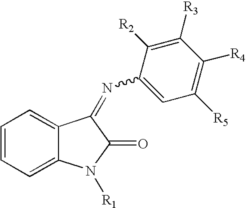

Substitution

| Compound | R1 | R2 | R3 | R4 | R5 |
|---|---|---|---|---|---|
| 1 | Ph | OMe | H | H | H |
| 2 | Ph | H | CF₃ | H | H |
| 3 | Ph | H | Me | H | H |
| 4 | Ph | H | Cl | H | H |
| 5 | Ph | H | H | CF₃ | H |
| 6 | Ph | H | H | Me | H |
| 7 | Ph | H | H | Cl | H |
| 8 | Ph | H | H | Br | H |
| 9 | Ph | H | H | F | H |
| 10 | Ph | H | H | OPh | H |
| 11 | Ph | H | H | OEt | H |
| 12 | Ph | H | H | OMe | H |
| 13 | Ph | H | Cl | H | Cl |
| 14 | Ph | H | Me | H | Me |
| 15 | Allyl | H | Cl | Cl | H |
| 16 | Allyl | H | Cl | H | Cl |
| 17 | Isopropyl | H | H | Br | H |

Key:
Ph = Phenyl
OMe = Methoxy
OEt = Ethoxy
Me = Methyl
OPh = Phenoxy

TABLE 1a
Chemical Structures of Compounds

| Compound | Structure |
|---|---|
| 18 | 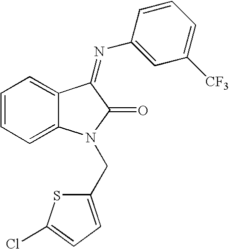 |
| 19 | 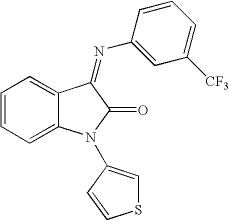 |
| 20 | 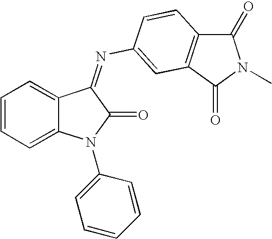 |
| 21 | 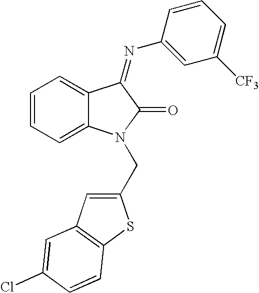 |
| 22 | 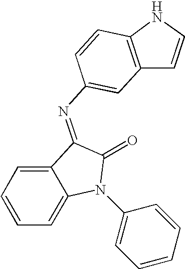 |
| 23 | 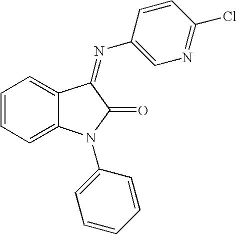 |
| 24 | 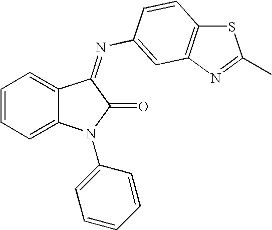 |
| 25 | 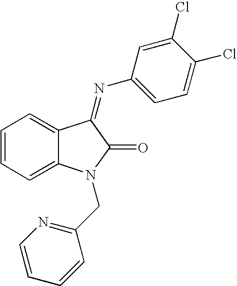 |
| 26 | 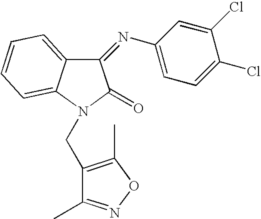 |
| 27 | 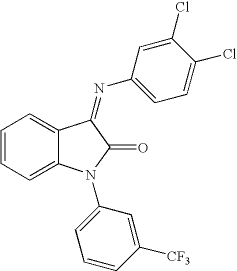 |
| 28 | 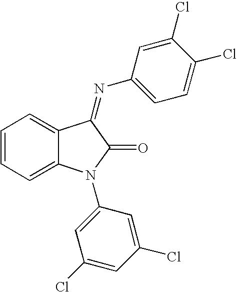 |
| 29 | 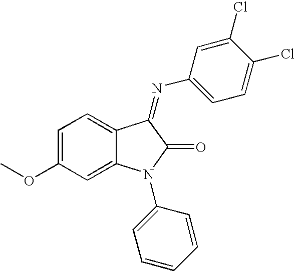 |
| 30 | 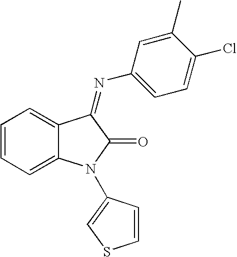 |
| 31 | 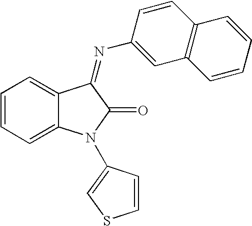 |
| 32 | 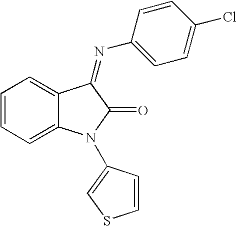 |
| 33 | 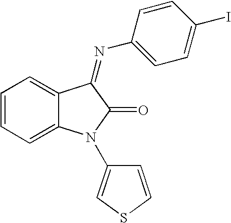 |
| 34 | 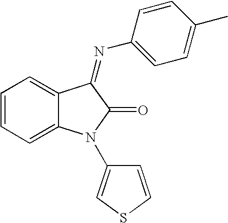 |
| 35 | 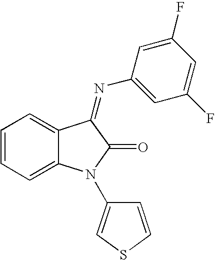 |
| 36 | 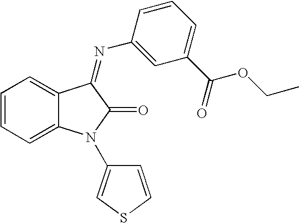 |
| 37 | 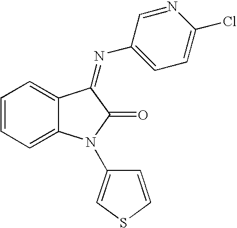 |
| 38 | 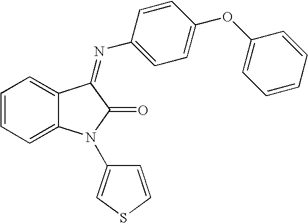 |
| 39 | 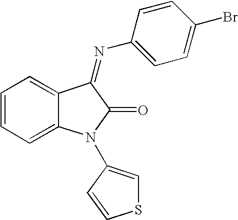 |
| 40 | 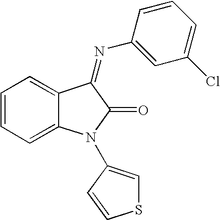 |
| 41 | 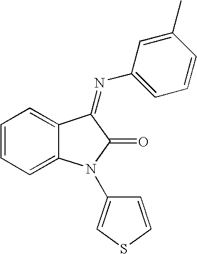 |
| 42 | 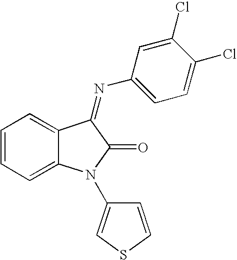 |
| 43 | 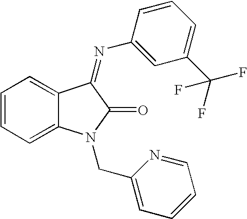 |
| 44 | 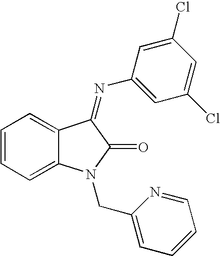 |
| 45 | 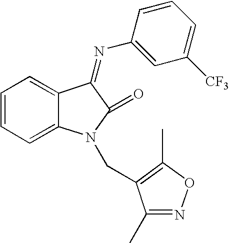 |
| 46 | 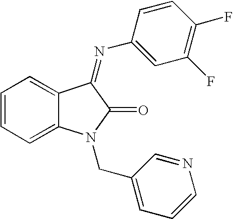 |
| 47 | 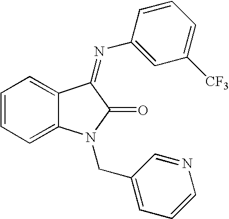 |
| 48 | 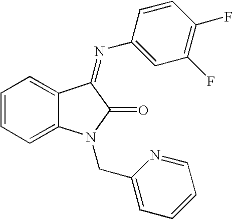 |
| 49 | 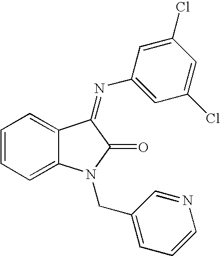 |
| 50 | 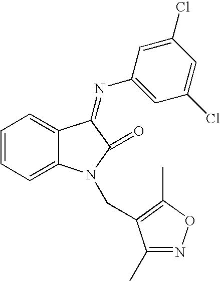 |
| 51 | 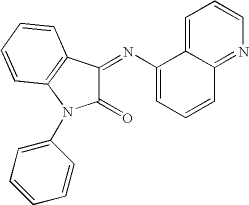 |
| 52 | 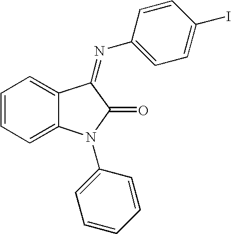 |
| 53 | 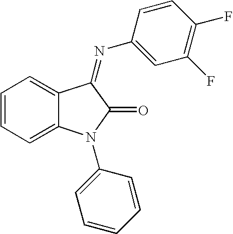 |
| 54 | 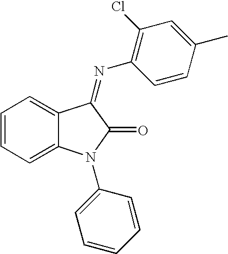 |
| 55 | 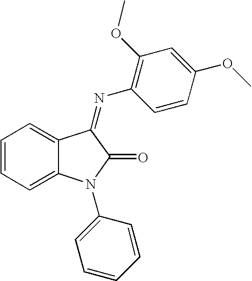 |
| 56 | 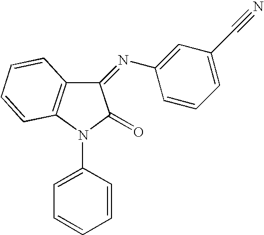 |
| 57 | 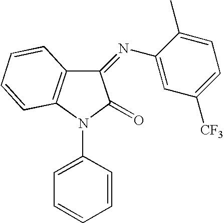 |
| 58 | 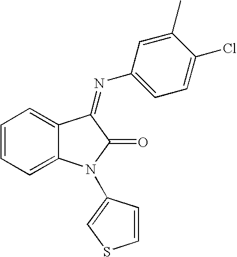 |
| 59 | 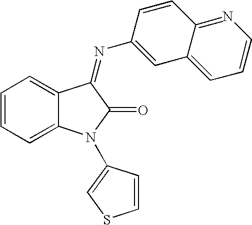 |
| 60 | 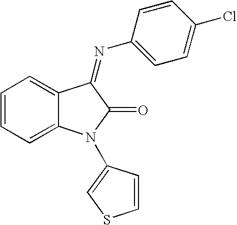 |
| 61 | 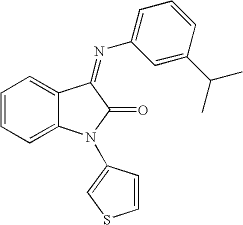 |
| 62 | 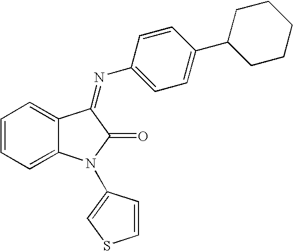 |
| 63 | 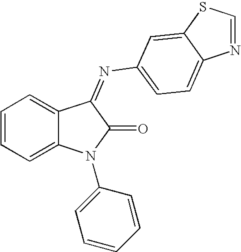 |
| 64 | 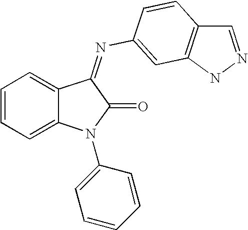 |
| 65 | 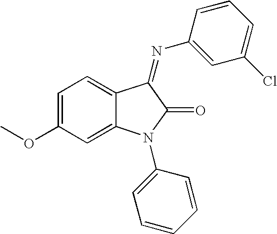 |
| 66 | 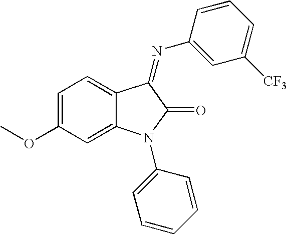 |
| 67 | 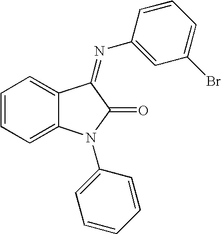 |
| 68 | 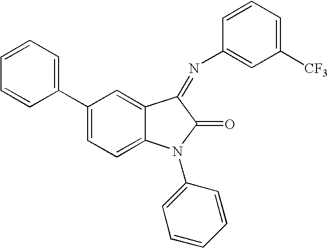 |
| 69 | 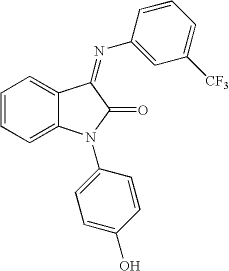 |
| 70 | 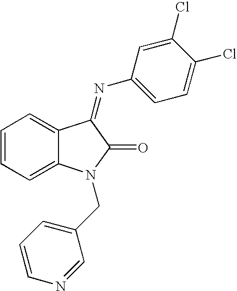 |

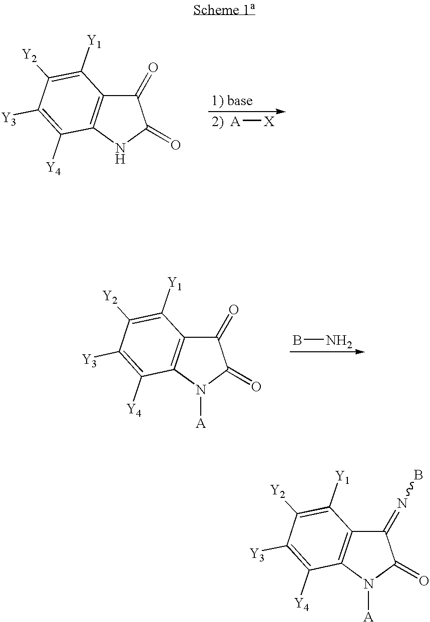 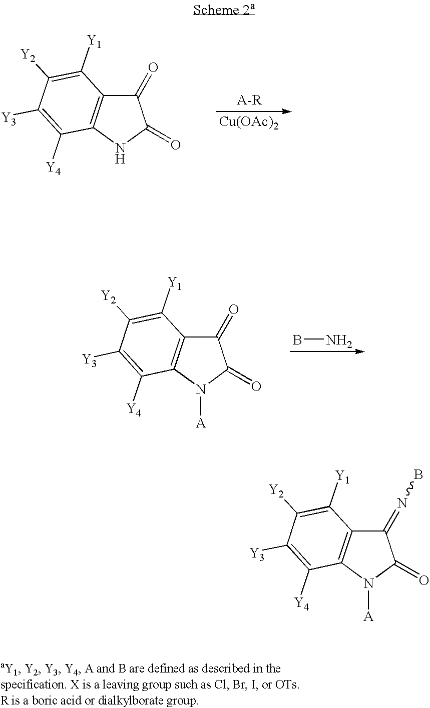 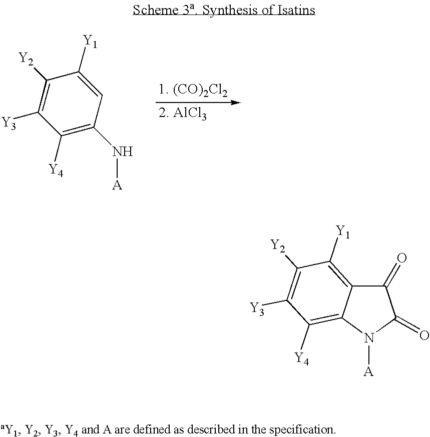 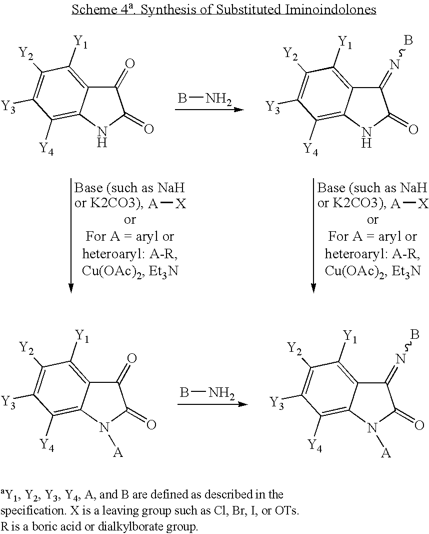 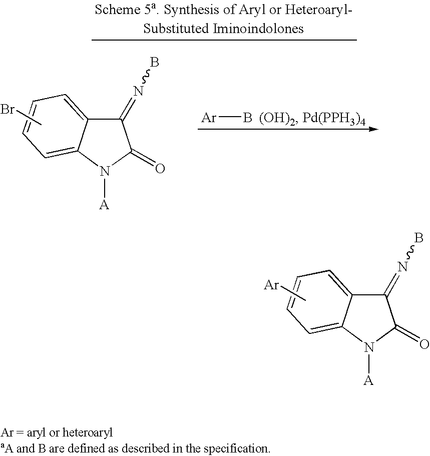 Pharmaceutical Compositions and Kits

As a specific embodiment of an oral composition of a compound of this invention, 100 mg of one of the compounds described herein is formulated with sufficient finely divided lactose to provide a total amount of 580 to 590 mg to fill a size O hard gel capsule.

The galanin-3 receptor antagonist compounds can be administered by any known means. For example, the compounds may be formulated as a capsule, suppository, cream, inhalant, or transdermal patch. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Optimal dosages to be administered may be determined by those skilled in the art, and will vary with the particular compound in use, the strength of the preparation, the mode of administration, and the advancement of the disease condition. Additional factors depending on the particular subject being treated will result in a need to adjust dosages, including subject age, weight, gender, diet, and time of administration. In the subject application a “therapeutically effective amount” is any amount of a compound which, when administered to a subject suffering from a disease against which the compounds are effective, causes reduction, remission, or regression of the disease. In the present application, a “subject” is a vertebrate, a mammal or a human.

The materials for use in the methods of the present invention are suited for preparation of kits produced in accordance with well known procedures. The kits may comprise containers, each with one or more of the various compounds utilized in the methods.

### EXAMPLES

### Example 1

### Neurite Outgrowth Assay

The cryopreserved mouse hippocampal neurons were plated into poly-L-lysine-coated 96 well plate (BD BioCoat) at 35,000 cells per well in Neurobasal/B27 medium (Invitrogen). 24 hr later the neurons were treated with HT-2157 at various concentrations and were subsequently fixed 48 hr post treatment.

PC12 sub-clone, Neuroscreen-1 (NS-1) cells were plated into collagen I-coated 96 well plate (BD BioCoat) at 5,000 cells per well in RPMI complete medium with 200 ng/mL NGF. 48 hr later NS-1 cells were treated with HT-2157 at various concentrations and were subsequently fixed 24 hr post treatment.

Following fixation, Cellomics's Neurite Outgrowth reagent kit was used to label cells by a primary antibody specific for neurons. The cell nuclei were labeled by Hoechst 33342. Fluorescently labeled cells were then imaged and analyzed using Cellomics's Neurite Outgrowth and Extended Neurite Outgrowth Bioapplications on the ArrayScan HCS Reader. Images for quantitative HCS analysis were collected on the ArrayScan HCS Reader using a 10× or a 20× microscope objective.

Quantitative Real-Time PCR (qPCR)

NS-1 cells were plated into collagen I-coated 6 well plate (BD BioCoat) at 150,000 cells per well. 48 hr post NGF treatment at 200 ng/mL NS-1 cells were treated with HT-2157 for 2 hr, 4 hr or 24 hr at concentrations as indicated followed by RNA isolation using Ambion's RNAqueous-4PCR kit. Reverse transcription reactions were performed with Taqman reverse transcription reagents (Applied Biosystems). qPCR was performed on a 7900 real-time PCR machine using Optical 96 well reaction plates (Applied Biosystems). Expression levels were normalized to mouse TBP transcript levels.

Results

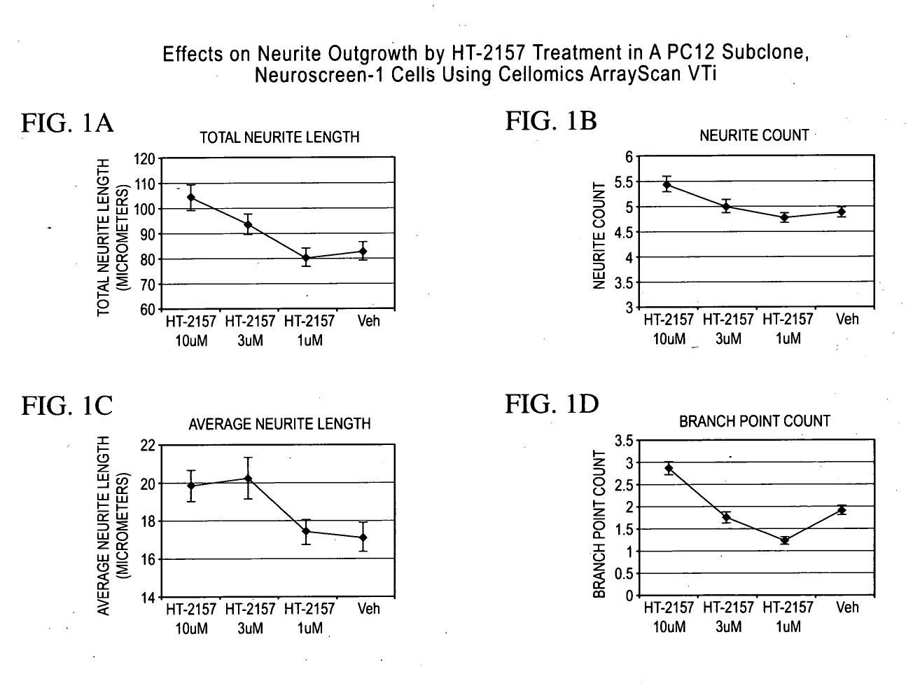

In HT-2157-treated NS-1 cells, the neurites were shown to increase in their count, length, and branch point in a dose-dependent manner. FIG. 1A shows an image of NS-1 cells acquired by a 1× objective lens on the ArrayScan HCS Reader. The top image is the raw image and the bottom image is the same field with a color overlay delineating the different features identified by the Extended Neurite Outgrowth BioApplication. In the color overlay image, cell nuclei are labeled in blue, cell bodies are labeled in red, neurites are labeled in green, and branch point in magenta. Rejected cells are labeled in orange. FIG. 1B to 1E show the plots of 4 output features generated from quantitative analysis of images of NS-1 cells by the Extended Neurite Outgrowth BioApplication. The data plotted is the mean value of each feature ±standard deviation from 2 wells per concentration of the compound. The definitions for the output features are as follows: neurite count: the number of neurites associated with the selected neurons; total neurite length: the total length of neurites for a selected neuron; average neurite length: the total neurite length divided by the neurite count for the selected neurons; and branch point: the junction of three neurite segments. Neurites are seen to increase in their length, count and branch point.

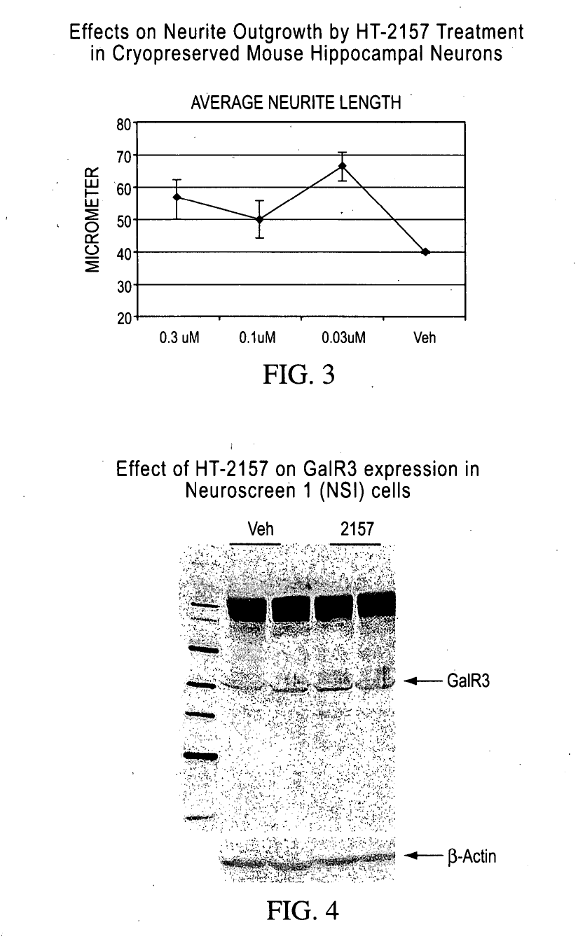

The similar enhancement of neurite outgrowth by HT-2157 was also observed in the cryopreserved mouse hippocampal neurons. FIG. 3A shows images of the cryopreserved mouse hippocampal neurons acquired by a 20× objective lens on the ArrayScan HCS Reader. The top image is the raw image and the bottom image is the same field with a green overlay tracing the neurites identified by the Neurite Outgrowth BioApplication. FIG. 3B shows the average neurite length analyzed by the Neurite Outgrowth BioApplication from the images of the mouse hippocampal neurons. The data plotted is the mean value ±standard deviation from 2 wells per concentration of the compound.

HT-2157 treatment significantly increased the neurite length in the cryopreserved mouse hippocampal neurons. Taken together, the enhancement on the neurite outgrowth of the NS-1 cells and the primary mouse hippocampal neurons by HT-2157 was demonstrated in this study.

In addition, the results indicate that HT-2157 exerted its roles in modulating neurite outgrowth through Hes5, a transcriptional repressor that negatively regulates neuronal differentiation. Hes5 expression was down-regulated by HT-2157 treatment at 2 hr and 4 hr in NS-1 cells. NS-1 cells treated by HT-2157 were subjected to quantitative real-time PCR analysis on Hes5, a transcriptional repressor that negatively regulates neuronal differentiation. Hes5 was down-regulated by HT-2157 treatment in a time- and dose-dependent manner, suggesting the enhancement of neurite outgrowth by HT-2157 is mediated through the control of neuronal differentiation progression.

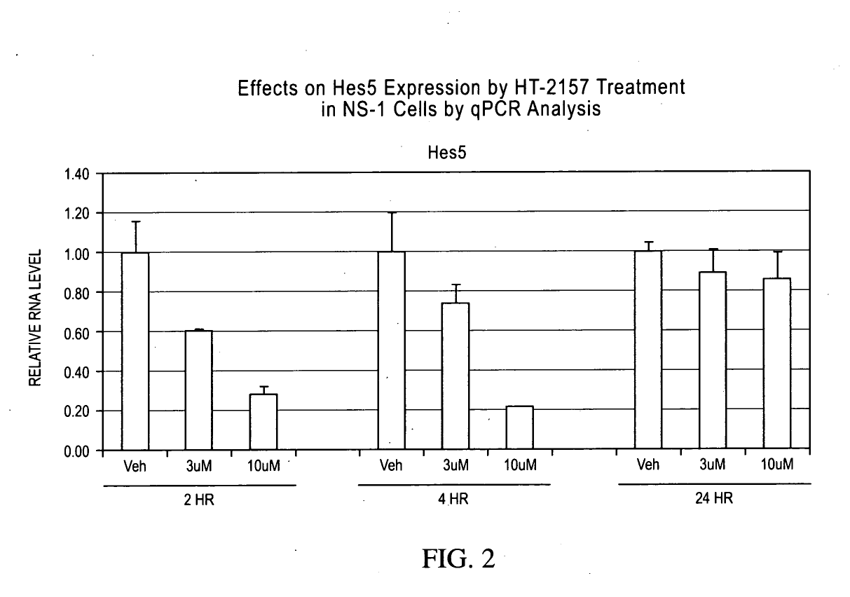

FIG. 2 shows the qPCR analysis of the effects on Hes5 expression by HT-2157 treatment in NS-1 cells. The data plotted is the mean value of the relative RNA level of the cells in 2 wells ±standard deviation.

### Example 2

### Neurite Outgrowth Assays

Neuronal Cell Culture

C57B1/6 or CD1 mouse embryonic (E17.5) hippocampal neurons were purchased from QBM Cell Science (University of Ottawa, Ontario, Canada). Neurons were cultured on poly-D-lysine coated 96 or 24 well plates in serum free Neurobasal medium supplemented with 2% B27, 500 μM L-glutamine, and 1 mM pyruvate. Cells were plated at a density of 20,000 per well on 96 well plates (for neurite outgrowth assays), and at 100,000 per well on 48 well plates (for qPCR analysis). For neurite outgrowth assays, neurons were cultured for 2 days and then stimulated for 24 hours. For gene-expression assays, neurons were grown for 8 days and then stimulated with HT-2157 or Vehicle.

NS1 Cell Culture

Neuroscreen 1 (NS1) Cells (Cellomics Inc.) were cultured on collagen type I coated 75 cm² plastic flasks (Biocoat, Becton Dickinson) in a humidified incubator at 37° C. in 5% CO₂. Cells were cultured in RPMI complete cell culture medium (Cambrex) supplemented with 10% heat-inactivated horse serum (Invitrogen), 5% heat-inactivated fetal bovine serum (Cellgro), and 2 mM L-glutamine (Cambrex). For expansion, the cells were trypsinized and split at 80% confluence. The cell culture media was changed every 2 to 3 days.

NS1 cells were stimulated with nerve growth factor to induce differentiation into a neuronal phenotype. NS1 cells were harvested as if they were being passaged and then counted using a Coulter counter (Becton Dickinson Coulter Z1). Cells were seeded in 96-well collagen I coated plates at a density of 2000 cells per well in volume of 200 μl. RPMI media was supplemented with 200 ng/ml nerve growth factor (NGFβ, Sigma). NS1 cells were incubated for 72 hours to allow differentiation to a neuronal phenotype. NGFβ was then diluted to 50 ng/ml and the cells were treated with siRNA or HT-2157, respectively.

Neurite Outgrowth Assay

Neurite outgrowth assays were performed using the Cellomics Arrayscan II Vti HCS scanner. Cells were stained using the HitKit™ HCS reagent kit (Cellomics) according to the manufactures specifications. The assay is based on immunofluorescence using an antibody that has been validated to specifically label both neurites and neuronal cell bodies. Briefly, cells were fixed in 3.7% formaldehyde and nuclei stained with Hoechst dye. Cells were then washed in neurite outgrowth buffer and neurites stained with Cellomics' proprietary primary antibody for neurite outgrowth high content screening. After 1 hour of incubation with the primary antibody, the cells were washed again and then incubated with fluorescently labeled secondary antibody solution for 1 hour. Antibody-stained 96-well plates were store at 4° C. in the dark until scanning. Plates were scanned using Cellomics ArrayScan II Vti HCS scanner. The neurite outgrowth assay uses two channels to carry out the scan. Channel 1 detects the Hoechst Dye and is used by the software to identify cells and for automated focusing. Channel 2 detects the FITC fluorescence of the secondary antibody and is used by the software to calculate all data generated in reference to neurites.

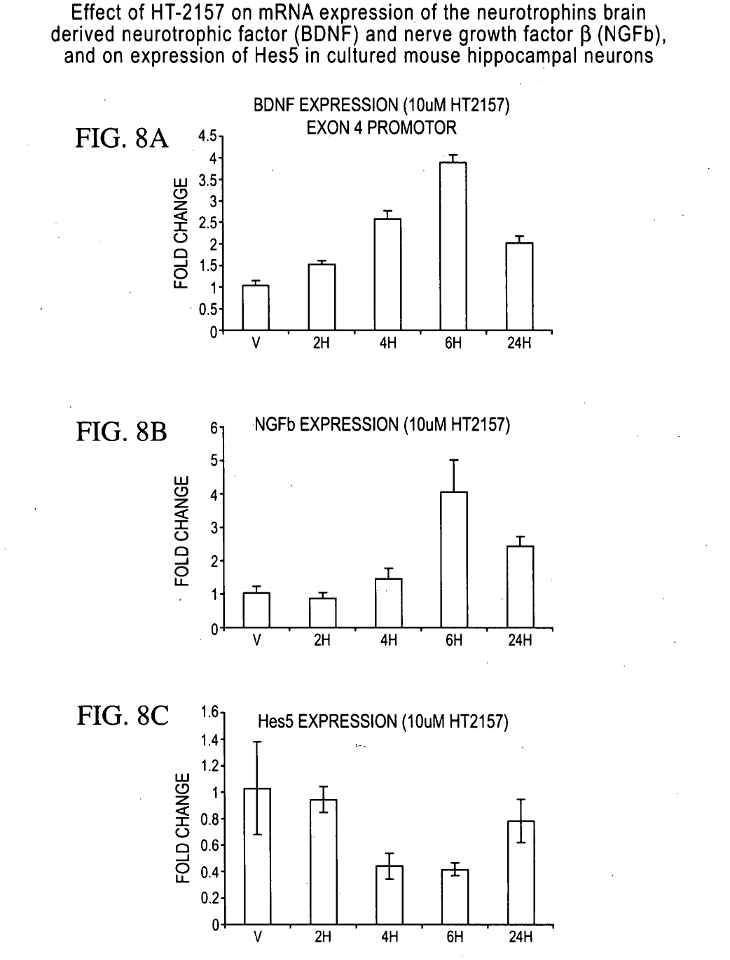

FIG. 7: shows the effect of HT-2157 on neurite outgrowth in NS1 cells. For each experiment, neurite outgrowth in a minimum of 100 cells was measured. Quantification of the effect of HT-2157 on neurite outgrowth in NS1 cells. 3 μM and 10 μM HT-2157 facilitates neurite outgrowth as indicated by an increase in: i) the number of neurites per cell (neurite count), ii) the total neurite length per cell, iii) the average neurite length per cell, iv) the number of neurite branch points per cell.

qPCR Analysis

RNA was isolated from cultured neurons at the indicated timepoint after HT-2157 treatment. Per well, one RNA preparation was performed using the QIAgen RNeasy kit (Qiagen) according to the manufacturer's specifications. cDNA was generated using TaqMan Reverse transcriptase kit (Applied Biosystems). 2 real-time PCR reactions per RNA/cDNA replication were performed using the ABI prism and SDS 2.1 software. ABI assays on demand (Applied Biosystems) were used to test the mRNA levels of BDNF, NGFβ, and Hes5. The average CT value for each cDNA sample was determined. Data was then normalized to TATA binding protein (TBP) and ΔCT values were determined. mRNA levels were normalized to a vehicle (0.2% DMSO) treated control group.

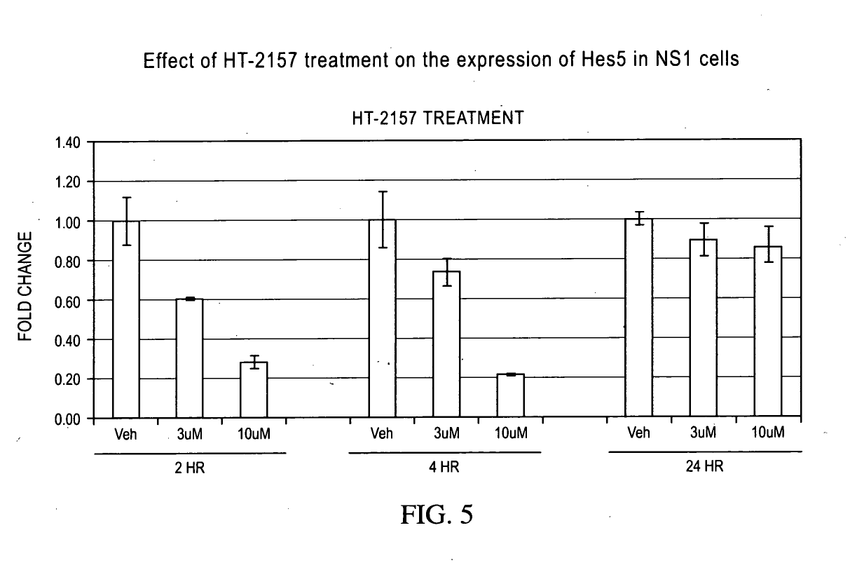

FIG. 4 shows the effect of HT-2157 on GalR3 expression in Neuroscreen 1 (NS1) cells. NS1 cells express the GalR3 receptor. Expression of GalR3 is not affected by HT-2157 treatment in NS1 cells.

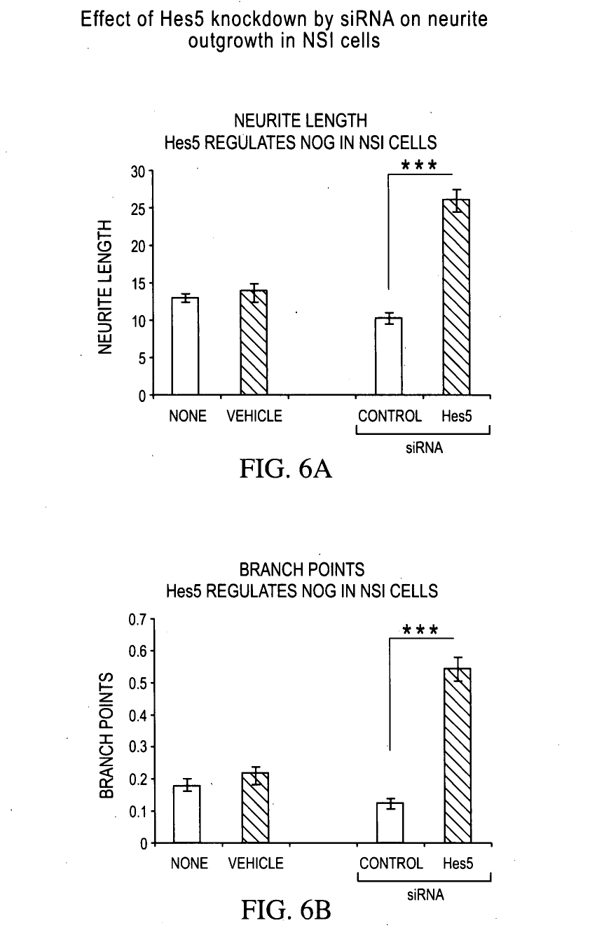

FIG. 5: shows the effect of HT-2157 treatment on the expression of Hes5 in NS1 cells. NS1 cells were treated with Vehicle (Veh) or HT-2157 for 2 hours, 4 hours, or 24 hours. When compared to vehicle treated controls, mRNA levels of Hes5 were reduced 2 hours and 4 hours after treatment with 3 or 10 μM HT-2157. Hes5 mRNA returned to baseline levels at 24 hours after treatment.

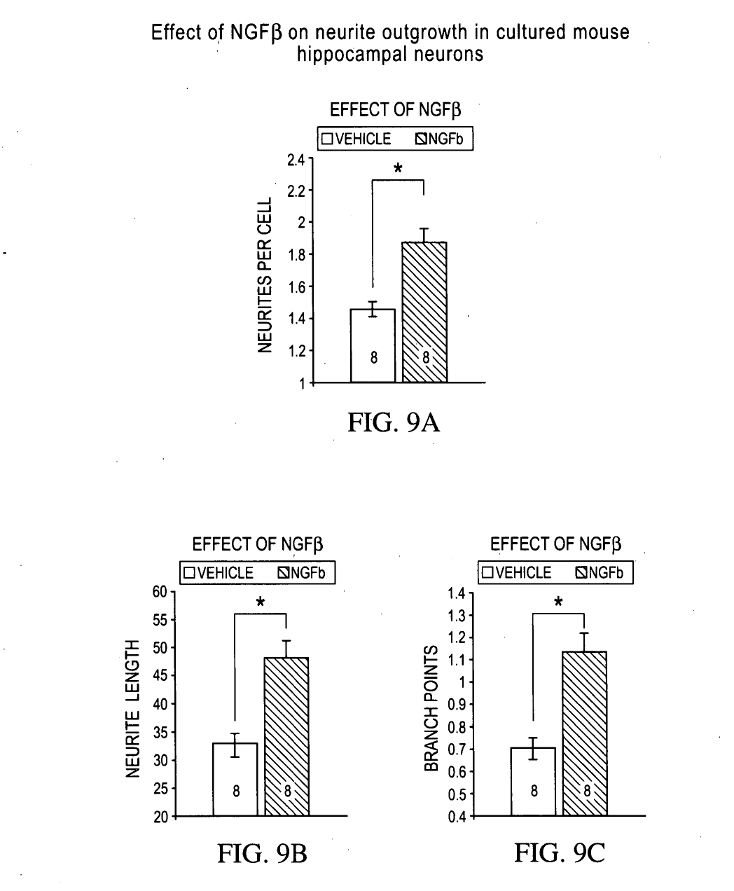

FIG. 8 shows the effect of HT-2157 on mRNA expression of the neurotrophins brain derived neurotrophic factor (BDNF) and nerve growth factor β (NGFb), and on expression of Hes5 in cultured mouse hippocampal neurons. The mean ±stdev of 2 experimental replications are shown. FIG. 8A shows the effect of HT-2157 on BDNF expression. Hippocampal neurons were treated with vehicle or 10 μM HT-2157 and BDNF mRNA levels determined by qPCR analysis. HT-2157 significantly increased BDNF mRNA levels in cultured neurons. FIG. 8B shows the effect of HT-2157 on NGFβ expression. Hippocampal neurons were treated with vehicle or 10 μM HT-2157 and NGFβ mRNA levels determined by qPCR analysis. HT-2157 significantly increased NGFβ mRNA levels in cultured neurons. FIG. 8C shows the effect of HT-2157 on Hes5 expression. Hippocampal neurons were treated with vehicle or 10M HT-2157 and Hes5 mRNA levels determined by qPCR analysis. HT-2157 significantly reduced Hes5 mRNA levels in cultured neurons, similar to its effect in NS1 cells (see FIG. 5). These results indicate that HT-2157 has a trophic effect on hippocampal neurons. BDNF and NGFβ have been implicated in neuronal survival and synaptic growth. Furthermore, HT-2157 inhibits Hes5 in both hippocampal neurons and NS1 cells.

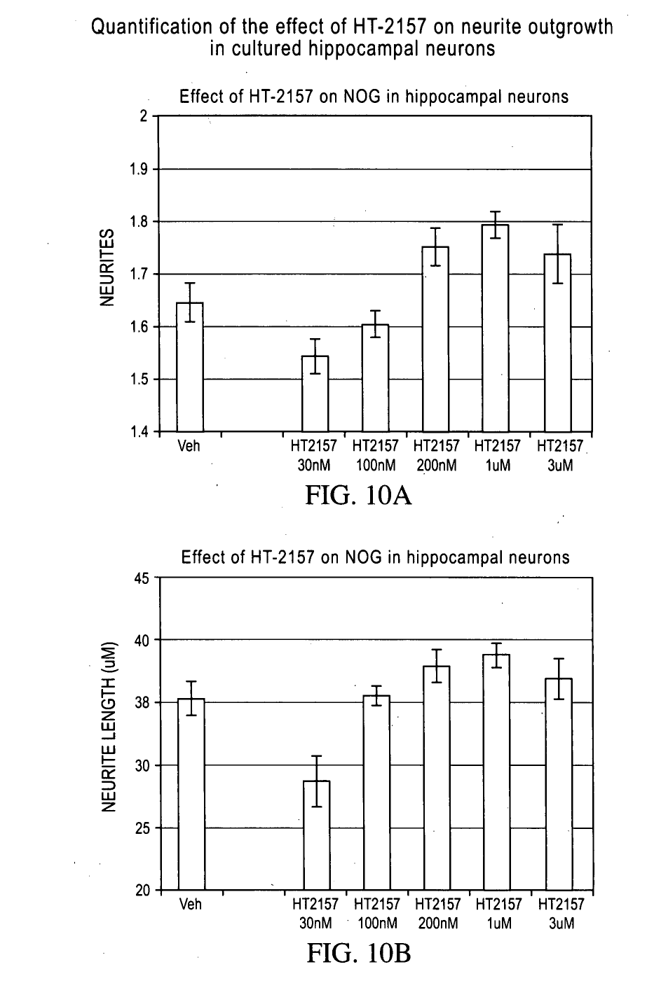

FIG. 9 shows the effect of NGFβ on neurite outgrowth in cultured mouse hippocampal neurons. The mean ±sem of 8 experimental replications are shown. For each experiment, neurite outgrowth in a minimum of 100 cells was measured. Hippocampal neuons were treated with 100 ng/ml NGFb for 24 hours and neurite growth measured in the Cellomics Arrayscan II. NGFβ enhances neurite outgrowth as evident by increased number of neurites per cell, increased neurite length, and increased branch points.

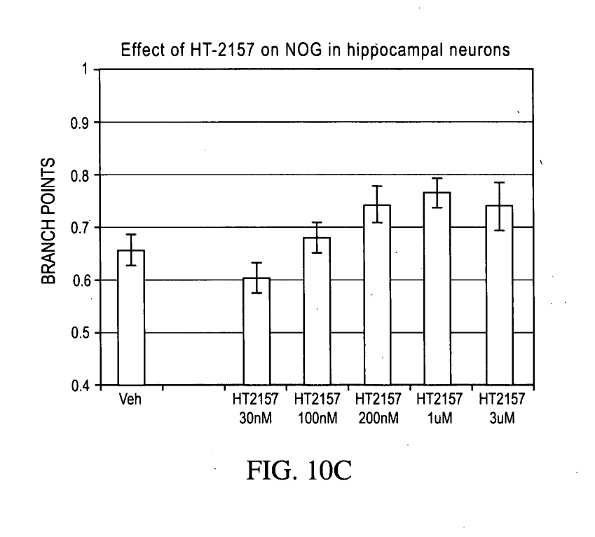

FIG. 10 is a quantification of the effect of HT-2157 on neurite outgrowth in cultured hippocampal neurons. The mean ±sem of 8 experimental replications (96-wells) per drug dose and 16 replication per vehicle are shown. For each experiment, neurite outgrowth in a minimum of 100 cells was measured. FIG. 10A shows the quantification of the effects of HT-2157 on neurite outgrowth in hippocampal neurons as determined by the effect on neurite number per cell. FIG. 10B shows the quantification of the effects of HT-2157 on neurite outgrowth in hippocampal neurons as determined by the effect on the total neurite length per cell. FIG. 10C shows the quantification of the effects of HT-2157 on neurite outgrowth in hippocampal neurons as determined by the effect on neurite branch points per cell. siRNA knockdown of Hes5

NS1 cells were primed to develop into a neuronal phenotype with NGFβ for 72 hours, and then transfected using 100 nM of siGENOME siRNA and Dharmafect 3. We used pools of siGENOME siRNA against Hes5 and a proprietary non-targeting control siRNA (Dharmacon, Lafayette, USA). Cells were incubated with siRNA or Dharmafect 3 only (vehicle) for 48 hours and then stained for neurite outgrowth assay as described.

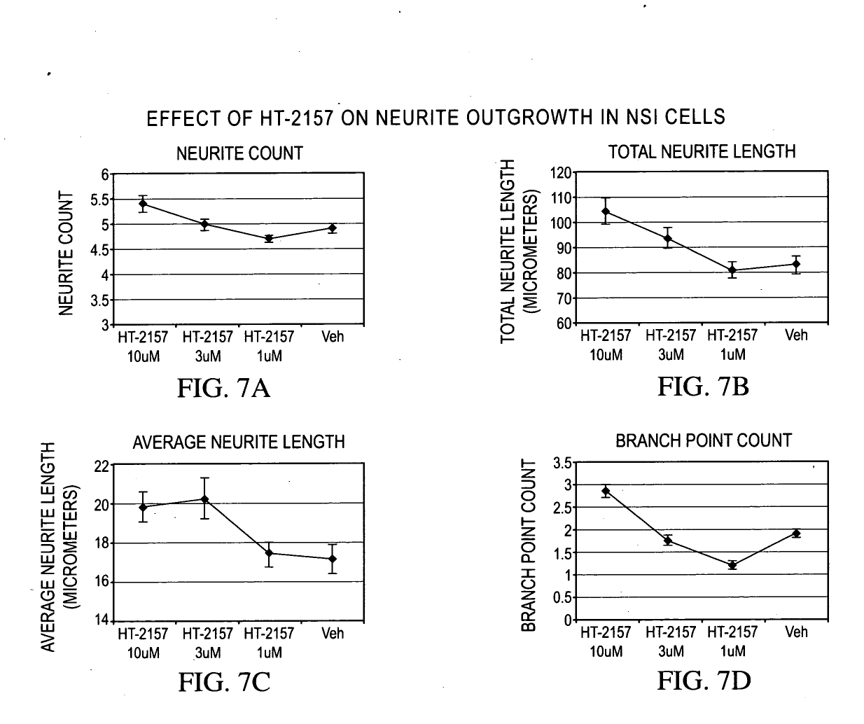

FIG. 6 shows the effect of Hes5 knockdown by siRNA on neurite outgrowth in NS1 cells. FIG. 6a shows neurite length in untreated NS1 cells, and in NS1 cells treated with Vehicle, control siRNA, Hes5 siRNA. NS1 cells treated with Hes5 siRNA had significantly longer neurites than vehicle or control siRNA treated NS1 cells.

FIG. 6b shows neurite branch points in untreated NS1 cells, and in NS1 cells treated with Vehicle, control siRNA, Hes5 siRNA. NS1 cells treated with Hes5 siRNA had significantly more neurite branch points than vehicle or control siRNA treated NS1 cells. p<0.001 for Hes5 vs. control siRNA.

This indicates that inhibition of Hes5 is sufficient to enhance neurite outgrowth in NS1 cells. Inhibition of Hes5 increases neurite length and the number of branch points per neurite. HT-2157 reduce Hes5 in NS1 cells and may thus facilitate neurite outgrowth.

Western Blotting

Cultured NS1 cells were homogenized in RIPA buffer (Upstate Biotechnology) containing proteinase inhibitors (Roche). Protein concentrations were determined using the Biorad DC protein assay kit (Biorad). 20 μg of protein-lysate were separated by SDS poly-acrylamide gel electrophoresis (SDS-PAGE) and blotted onto nylon membranes. Western blots were blocked with 5% non-fat dry milk in Tris-buffered saline containing 0.05% Tween 20 (TBS-T) and the primary antibodies applied at 4° Celsius over night. Blots were probed with horseradish peroxidase (HRP) coupled secondary antibodies at room temperature for 1 h, and developed using the SuperSignal® West Pico Chemiluminescent Substrate (Pierce). We used a polyclonal antibody against GalR3 (Alpha Diagnostics). Blots were normalized to β-actin (Sigma).

Statistical Analysis

The means and standard deviations of several experimental replications (48-well or 96-well) were determined. Data were analyzed by student's t-test or one-way ANOVA. Unless indicated otherwise, values shown in the graphs represent mean ±SD.

All publications, patent and patent applications mentioned in this specification are incorporated herein by reference to the same extent as if each individual publication, patent or patent application was specifically and individually incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for modulating neurite outgrowth in an animal by the administration of a galanin-3 receptor antagonist to an animal.

2. The method of claim 1 wherein said animal is a human

3. The method of claim 2 wherein said animal has a neurodegenerative disease or condition.

4. The method of claim 2 wherein said animal has neuronal stem cell manipulation.

5. The method of claim 1 wherein the galanin-3 receptor antagonist inhibitor is HT-2157 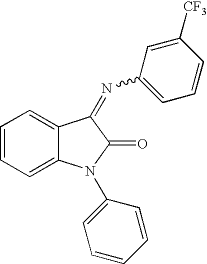
the E/Z isomers or mixtures thereof.

6. The method of claim 1 wherein said galanin-3 receptor antagonist is administered once.

7. The method of claim 1 wherein said galanin-3 receptor antagonist is administered repeatedly over a period of time.

8. The method of claim 1 wherein the galanin-3 receptor antagonist has the structure: 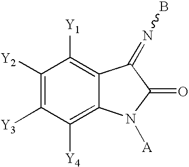
wherein each of Y₁, Y₂, Y₃, and Y₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, or C₅-C₇ cycloalkenyl; —F, —Cl, —Br, or —I; —NO₂; —N₃; —CN; —OR₄, —SR₄, —OCOR₄, —COR₄, —NCOR₄, —N(R₄)₂, —CON(R₄)₂, or —COOR₄; aryl or heteroaryl; or any two of Y₁, Y₂, Y₃ and Y₄ present on adjacent carbon atoms can constitute a methylenedioxy group;
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl;
wherein A is A′, straight chained or branched C₁-C₇ alkyl, aryl, heteroaryl, aryl(C₁-C₆)alkyl or heteroaryl(C₁-C₆)alkyl;
wherein A′ is 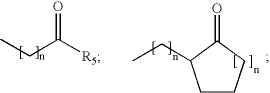
wherein R₁ and R₂ are each independently —H, straight chained or branched C₁-C₇ alkyl, —F, —Cl, — 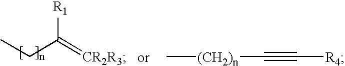
Br, —I, —NO₂, or —CN;
wherein R₃ is —H, straight chained or branched C₁-C₇ alkyl, —F, —Cl, —Br, —I, —NO₂, —CN, —OR₆, aryl or heteroaryl;
wherein R₅ is straight chained or branched C₁-C₇ alkyl, —N(R₄)₂, —OR₆ or aryl;
wherein R₆ is straight chained or branched C₁-C₇ alkyl or aryl;
wherein B is aryl, or heteroaryl; provided however, if B is aryl or heteroaryl the carbon atom or carbon atoms ortho to the nitrogen atom of the imine bond may only be substituted with one or more of the following: —H, —F, —Cl, —Br, —I, —CN, methyl, ethyl or methoxy;
wherein each n is independently an integer from 1 to 4 inclusive;
wherein the compound is a pure Z imine isomer, a pure E imine isomer, or a mixture of Z and E imine isomers;
or a pharmaceutically acceptable salt thereof.

9. The method of claim 1 wherein the galanin-3 receptor antagonist has the structure: 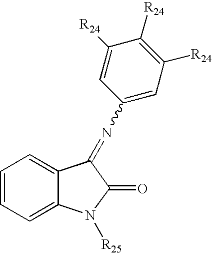
wherein each R₂₄ is independently one or more of the following: H, F, Cl, Br, I, CF₃ or OCH₃;
wherein R₂₅ is methyl, ethyl, allyl or phenyl and the phenyl is optionally substituted with a F, Cl, Br, CF₃, or OR₄; and
wherein each R₄ is indenpendently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl.

10. The method of claim 1 wherein the a galanin-3 receptor antagonist compound has the structure. 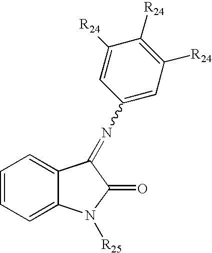
wherein each R₂₄ is independently one or more of the following: H, F, Cl, Br, I, CF₃ or OCH₃;
wherein R₂₅ is methyl, ethyl, allyl or phenyl and the phenyl is optionally substituted with a F, Cl, Br, CF₃, or OR₄; and
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl.

11. A method of treating a subject in need of treatment for a nerve cellular injury and/or trauma which comprises administering to the subject a galanin-3 receptor antagonist.

12. The method of claim 11 wherein said animal has neuronal stem cell manipulation.

13. The method of claim 11 wherein the galanin-3 receptor antagonist inhibitor is HT-2157 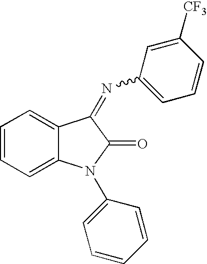
the E/Z isomers or mixtures thereof.

14. The method of claim 11 wherein said galanin-3 receptor antagonist is administered once.

15. The method of claim 11 wherein said galanin-3 receptor antagonist is administered repeatedly over a period of time.

16. The method of claim 11 wherein the galanin-3 receptor antagonist has the structure: 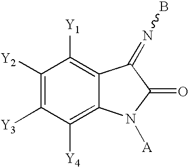
wherein each of Y₁, Y₂, Y₃, and Y₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, or C₅-C₇ cycloalkenyl; —F, —Cl, —Br, or —I; —NO₂; —N₃; —CN; —OR₄, —SR₄, —OCOR₄, —COR₄, —NCOR₄, —N(R₄)₂, —CON(R₄)₂, or —COOR₄; aryl or heteroaryl; or any two of Y₁, Y₂, Y₃ and Y₄ present on adjacent carbon atoms can constitute a methylenedioxy group;
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl;
wherein A is A′, straight chained or branched C₁-C₇ alkyl, aryl, heteroaryl, aryl(C₁-C₆)alkyl or heteroaryl(C₁-C₆)alkyl;
wherein A′ is 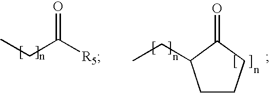
wherein R₁ and R₂ are each independently —H, straight chained or branched C₁-C₇ alkyl, —F, —Cl, — 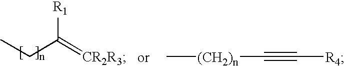
Br, —I, —NO₂, or —CN;
wherein R₃ is —H, straight chained or branched C₁-C₇ alkyl, —F, —Cl, —Br, —I, —NO₂, —CN, —OR₆, aryl or heteroaryl;
wherein R₅ is straight chained or branched C₁-C₇ alkyl, —N(R₄)₂, —OR₆ or aryl;
wherein R₆ is straight chained or branched C₁-C₇ alkyl or aryl;
wherein B is aryl, or heteroaryl; provided however, if B is aryl or heteroaryl the carbon atom or carbon atoms ortho to the nitrogen atom of the imine bond may only be substituted with one or more of the following: —H, —F, —Cl, —Br, —I, —CN, methyl, ethyl or methoxy;
wherein each n is independently an integer from 1 to 4 inclusive;
wherein the compound is a pure Z imine isomer, a pure E imine isomer, or a mixture of Z and E imine isomers;
or a pharmaceutically acceptable salt thereof.

17. The method of claim 11 wherein the galanin-3 receptor antagonist has the structure: 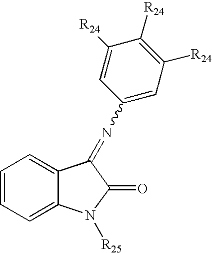
wherein each R₂₄ is independently one or more of the following: H, F, Cl, Br, I, CF₃ or OCH₃;
wherein R₂₅ is methyl, ethyl, allyl or phenyl and the phenyl is optionally substituted with a F, Cl, Br, CF₃, or OR₄; and
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl.

18. The method of claim 11 wherein the a galanin-3 receptor antagonist compound has the structure: 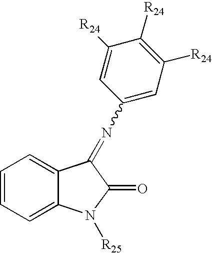
wherein each R₂₄ is independently one or more of the following: H, F, Cl, Br, I, CF₃ or OCH₃;
wherein R₂₅ is methyl, ethyl, allyl or phenyl and the phenyl is optionally substituted with a F, Cl, Br, CF₃, or OR₄; and
wherein each R₄ is independently —H; straight chained or branched C₁-C₇ alkyl, monofluoroalkyl or polyfluoroalkyl; straight chained or branched C₂-C₇ alkenyl or alkynyl; C₃-C₇ cycloalkyl, C₅-C₇ cycloalkenyl, aryl or aryl(C₁-C₆)alkyl.

19. The method of claim 11 wherein the nerve cellular injury or trauma is primary nervous system injury selected from the group consisting of closed head injuries and blunt trauma, penetrating trauma, hemorrhagic stroke, ischemic stroke, glaucoma, cerebral ischemia, or damages caused by surgery such as tumor excision.

20. The method of claim 11 wherein the nerve cellular injury or trauma is primary diseases or disorders of the central or peripheral nervous system selected from the group consisting of diabetic neuropathy and amyotrophic lateral sclerosis (ALS).

21. The method of claim 11 wherein the nerve cellular injury or trauma is peripheral nerve injuries and peripheral or localized neuropathies selected from the group consisting of porphyria, acute sensory neuropathy, chronic ataxic neuropathy, complications of various drugs and toxins, amyloid polyneuropathies, adrenomyeloneuropathy, or giant axonal neuropathy

22. The method of claim 11 wherein the nerve cellular injury or trauma is spinal chord trauma.

23. The method of claim 11 further comprising treating stem cells or neuronal progenitor cells prior to the cells being administered to the patient by implantation at the site of neuronal degeneration.

24. A kit for the treatment of neural cellular injury and/or trauma comprising a galanin-3 receptor antagonist.
